(19) United States
(12) Patent Application Publication
(10) Pub. No.: US 2025/0140378 A1
(43) Pub. Date: May 1, 2025

(54) USER PHYSIOLOGICAL RESPONSE PREDICTION AND MANAGEMENT SYSTEMS

(71) Applicant: ABBOTT DIABETES CARE INC., Alameda, CA (US)

(72) Inventors: Erwin S. Budiman, Fremont, CA (US); Louis G. Pace, Auburn, CA (US); Vivek S. Rao, Alameda, CA (US); Peter G. Robinson, Alamo, CA (US); Marc B. Taub, Mountain View, CA (US); Peter M. Voit, Dublin, CA (US); Chung-Che Wang, Palo Alto, CA (US); Leilani Lin Berchesi, Alameda, CA (US); Shridhara Alva Karinka, Pleasanton, CA (US); Jacob Sunol Canadas, Alameda, CA (US)

(21) Appl. No.: 18/930,330

(22) Filed: Oct. 29, 2024

Related U.S. Application Data
(60) Provisional application No. 63/594,332, filed on Oct. 30, 2023.

(51) Int. Cl. G16H 20/60; A61M 5/172; G06V 20/52; G06V 20/68

(52) U.S. Cl. CPC G16H 20/60; A61M 5/1723; G06V 20/52; G06V 20/68; A61M 2205/3306; A61M 2230/201

(57) ABSTRACT

A system for predicting and/or managing physiological responses is configurable to (i) access one or more images depicting one or more consumables able to influence a physiological condition of a user, the one or more images being associated with one or more timepoints; (ii) access user state information associated with the user; (iii) determine a predicted physiological response to consumption of the one or more consumables by the user based on at least the one or more images and the user state information; and (iv) present an output based on the predicted physiological response to the user via a user interface.

### CROSS-REFERENCE TO RELATED APPLICATION(S)

This application claims priority to U.S. Provisional Patent Application No. 63/594,332, filed on Oct. 30, 2023, and entitled “USER PHYSIOLOGICAL RESPONSE PREDICTION AND MANAGEMENT USING EXTENDED REALITY SYSTEMS”, the entirety of which is incorporated herein by reference for all purposes.

### BACKGROUND

Individuals with diabetes face challenges in maintaining stable blood sugar levels. Managing food and beverage intake is an important aspect of diabetes management. For instance, carbohydrates can have a significant impact on blood sugar levels. People with diabetes thus often need to carefully monitor their carbohydrate intake, as excessive consumption of carbohydrates can lead to spikes in blood sugar levels. However, accurately estimating the carbohydrate content of meals can be challenging, particularly when eating food not prepared by oneself or when facing complex food items.

The glycemic index (GI) is a measure of how quickly a particular food raises blood sugar levels. Foods with a high GI value can cause rapid spikes in blood sugar levels, while those with a low GI value can cause a more gradual increase in blood sugar levels. Knowledge of GI values associated with meals allows individuals with diabetes to make informed choices. However, it can be difficult to consistently track and remember the GI values of various foods, especially in social settings or when faced with unfamiliar ingredients. Furthermore, how quickly a particular food affects blood sugar levels can be influenced by other factors, such as stress state, disease state, blood alcohol level, medication usage, physical activity level, etc. Individuals can experience difficulty in tracking such factors and inferring the influence of such factors on blood sugar levels (or change in blood sugar levels).

Maintaining appropriate portion sizes can be important for people with diabetes to manage their blood sugar levels. However, accurately estimating portion sizes can be challenging, and overeating or undereating can have significant consequences for blood sugar control. External factors, such as larger portions served at restaurants or social pressures to indulge, can further complicate portion control efforts for individuals with diabetes.

There exists a need for improved methods and techniques to enable individuals with diabetes to maintain stable blood sugar levels and achieve desirable health outcomes.

The subject matter claimed herein is not limited to embodiments that operate only in environments such as those described above. Rather, this background is only provided to illustrate one example technology area where some embodiments described herein may be practiced.

### BRIEF SUMMARY

In some aspects, the techniques described herein relate to a system, comprising: one or more processors; and one or more computer-readable recording media that store instructions that are executable by the one or more processors to configure the system to: access one or more images depicting one or more consumables, the consumption of which is able to influence a physiological condition of a user, the one or more images being associated with one or more timepoints; access user state information associated with the user; determine a predicted physiological response to consumption of the one or more consumables by the user based on at least the one or more images and the user state information; and present an output based on the predicted physiological response to the user via a user interface.

In some aspects, the techniques described herein relate to a system, wherein the output comprises a representation of the predicted physiological response.

In some aspects, the techniques described herein relate to a system, wherein the output comprises a predicted consequence of consuming the one or more consumables.

In some aspects, the techniques described herein relate to a system, wherein the output comprises a recommended action of the user.

In some aspects, the techniques described herein relate to a system, wherein the output is based on a comparison of the predicted physiological response and an alternative predicted physiological response associated with an alternative course of action of the user.

In some aspects, the techniques described herein relate to a system, wherein the system comprises one or more image sensors configured to capture the one or more images during operation of the system by the user.

In some aspects, the techniques described herein relate to a system, wherein the system comprises an extended reality system.

In some aspects, the techniques described herein relate to a system, wherein the one or more consumables comprise at least one of one or more food items or one or more beverage items.

In some aspects, the techniques described herein relate to a system, wherein the physiological condition comprises blood glucose level.

In some aspects, the techniques described herein relate to a system, wherein the physiological condition comprises a metabolic state.

In some aspects, the techniques described herein relate to a system, wherein the physiological condition comprises one or more health metrics.

In some aspects, the techniques described herein relate to a system, wherein the user state information is determined based upon image data, sensor data, or user input.

In some aspects, the techniques described herein relate to a system, wherein the user state information indicates at least one of current or historic blood glucose level for the user.

In some aspects, the techniques described herein relate to a system, wherein the current or historic blood glucose level for the user is obtained via continuous glucose monitoring.

In some aspects, the techniques described herein relate to a system, wherein the user state information further indicates at least one of a ketone level, a lactate level, or an alcohol level for the user.

In some aspects, the techniques described herein relate to a system, wherein the user state information indicates one or more therapeutics administered to the user within one or more threshold temporal proximities to the one or more timepoints.

In some aspects, the techniques described herein relate to a system, wherein the user state information is determined based on image data that captures one or more physical components associated with the one or more therapeutics administered to the user.

In some aspects, the techniques described herein relate to a system, wherein the one or more therapeutics do not comprise insulin.

In some aspects, the techniques described herein relate to a system, wherein the one or more threshold temporal proximities are determined based on one or more therapeutic classifications of the one or more therapeutics.

In some aspects, the techniques described herein relate to a system, wherein the user state information indicates at least one of a stress state or a disease state of the user.

In some aspects, the techniques described herein relate to a system, wherein the user state information indicates a blood alcohol level of the user.

In some aspects, the techniques described herein relate to a system, wherein the user state information indicates one or more financial preferences or financial states of the user.

In some aspects, the techniques described herein relate to a system, wherein the user state information indicates one or more anticipated activities of the user.

In some aspects, the techniques described herein relate to a system, wherein the user state information indicates one or more user preferences associated with a lifestyle plan for the user.

In some aspects, the techniques described herein relate to a system, wherein the predicted physiological response comprises at least one of a predicted blood glucose level or blood glucose curve.

In some aspects, the techniques described herein relate to a system, wherein the predicted physiological response comprises a predicted metabolic state.

In some aspects, the techniques described herein relate to a system, wherein the predicted physiological response comprises a predicted effect on one or more health metrics.

In some aspects, the techniques described herein relate to a system, wherein the user interface comprises at least one of a display, a speaker, or a haptic feedback device of the system.

In some aspects, the techniques described herein relate to a system, wherein the output comprises at least one of a graphical representation, an audio representation, or a haptic representation.

In some aspects, the techniques described herein relate to a system, wherein the instructions are executable by the one or more processors to further configure the system to automatically update a therapeutic delivery configuration of a therapeutic delivery device based upon the predicted physiological response.

In some aspects, the techniques described herein relate to a system, wherein the therapeutic delivery device comprises an insulin pump operably connected to the system.

In some aspects, the techniques described herein relate to a system, wherein the therapeutic delivery configuration comprises one or more of: bolus insulin configuration, basal insulin configuration, or insulin delivery schedule.

In some aspects, the techniques described herein relate to a system, wherein determining the predicted physiological response further utilizes historical information associated with the user.

In some aspects, the techniques described herein relate to a system, wherein determining the predicted physiological response comprises determining one or more consumable characteristics associated with the one or more consumables and determining the predicted physiological response based on the one or more consumable characteristics.

In some aspects, the techniques described herein relate to a system, wherein the predicted physiological response is determined directly from the one or more images and the user state information without determining one or more consumable characteristics associated with the one or more consumables.

In some aspects, the techniques described herein relate to a system, wherein the instructions are executable by the one or more processors to further configure the system to: access one or more subsequent images depicting at least part of the one or more consumables at one or more subsequent timepoints; determine an updated predicted physiological response based on at least the one or more subsequent images; and present an updated output based on the updated predicted physiological response.

In some aspects, the techniques described herein relate to a system, wherein the updated predicted physiological response is further based on updated user state information associated with the user.

In some aspects, the techniques described herein relate to a system, wherein the instructions are executable by the one or more processors to further configure the system to: access one or more subsequent images depicting at least part of the one or more consumables at one or more subsequent timepoints; determine a consumption metric based on at least the one or more subsequent images; and update user state information based on the consumption metric.

In some aspects, the techniques described herein relate to a system, comprising: one or more processors; and one or more computer-readable recording media that store instructions that are executable by the one or more processors to configure the system to: receive an indication of one or more consumables from one or more remote systems, consumption of the one or more consumables being able to influence a physiological condition of a user; access user state information associated with the user; determine a predicted physiological response to consumption of the one or more consumables by the user based on at least the indication of the one or more consumables and the user state information; and at least one of (i) present an output based on the predicted physiological response to the user via a user interface or (ii) automatically update a therapeutic delivery configuration of a therapeutic delivery device based upon the predicted physiological response.

In some aspects, the techniques described herein relate to a system, wherein the output comprises a representation of the predicted physiological response.

In some aspects, the techniques described herein relate to a system, wherein the output comprises a predicted consequence of consuming the one or more consumables.

In some aspects, the techniques described herein relate to a system, wherein the output comprises a recommended action of the user.

In some aspects, the techniques described herein relate to a system, wherein the output is based on a comparison of the predicted physiological response and an alternative predicted physiological response associated with an alternative course of action of the user.

In some aspects, the techniques described herein relate to a system, wherein the system comprises an extended reality system.

In some aspects, the techniques described herein relate to a system, wherein the one or more consumables comprise at least one of one or more food items or one or more beverage items.

In some aspects, the techniques described herein relate to a system, wherein the physiological condition comprises blood glucose level.

In some aspects, the techniques described herein relate to a system, wherein the physiological condition comprises a metabolic state.

In some aspects, the techniques described herein relate to a system, wherein the physiological condition comprises one or more health metrics.

In some aspects, the techniques described herein relate to a system, wherein the user state information is determined based upon at least one of image data, sensor data, or user input.

In some aspects, the techniques described herein relate to a system, wherein the user state information indicates at least one of current or historic blood glucose level for the user.

In some aspects, the techniques described herein relate to a system, wherein the current or historic blood glucose level for the user is obtained via continuous glucose monitoring.

In some aspects, the techniques described herein relate to a system, wherein the user state information further indicates at least one of a ketone level, a lactate level, or an alcohol level for the user.

In some aspects, the techniques described herein relate to a system, wherein the user state information indicates one or more therapeutics administered to the user.

In some aspects, the techniques described herein relate to a system, wherein the user state information is determined based on image data that captures one or more physical components associated with the one or more therapeutics administered to the user.

In some aspects, the techniques described herein relate to a system, wherein the one or more therapeutics do not comprise insulin.

In some aspects, the techniques described herein relate to a system, wherein the user state information indicates at least one of a stress state or a disease state of the user.

In some aspects, the techniques described herein relate to a system, wherein the user state information indicates a blood alcohol level of the user.

In some aspects, the techniques described herein relate to a system, wherein the user state information indicates at least one of one or more financial preferences or one or more financial states of the user.

In some aspects, the techniques described herein relate to a system, wherein the user state information indicates one or more anticipated activities of the user.

In some aspects, the techniques described herein relate to a system, wherein the user state information indicates one or more user preferences associated with a lifestyle plan for the user.

In some aspects, the techniques described herein relate to a system, wherein the predicted physiological response comprises at least one of a predicted blood glucose level or blood glucose curve.

In some aspects, the techniques described herein relate to a system, wherein the predicted physiological response comprises a predicted metabolic state.

In some aspects, the techniques described herein relate to a system, wherein the predicted physiological response comprises a predicted effect on one or more health metrics.

In some aspects, the techniques described herein relate to a system, wherein the user interface comprises at least one of a display, a speaker, or a haptic feedback device of the system.

In some aspects, the techniques described herein relate to a system, wherein the output comprises at least one of a graphical representation, an audio representation, or a haptic representation.

In some aspects, the techniques described herein relate to a system, wherein the therapeutic delivery device comprises an insulin pump operably connected to the system.

In some aspects, the techniques described herein relate to a system, wherein the therapeutic delivery configuration comprises one or more of: bolus insulin configuration, basal insulin configuration, or insulin delivery schedule.

In some aspects, the techniques described herein relate to a system, wherein determining the predicted physiological response further utilizes historical information associated with the user.

In some aspects, the techniques described herein relate to a system, comprising: one or more processors; and one or more computer-readable recording media that store instructions that are executable by the one or more processors to configure the system to: access one or more images depicting one or more consumables, the consumption of which is able to influence a physiological condition of a user; access user state information associated with the user; output a prompt for the user to provide user input indicating the one or more consumables to be consumed by the user; determine a predicted physiological response to consumption of the one or more consumables by the user based on at least the one or more images, the user state information, and the user input; and output the predicted physiological response.

In some aspects, the techniques described herein relate to a system, wherein the instructions are executable by the one or more processors to further configure the system to: receive user input indicating a user-estimated physiological response to consumption of the one or more consumables by the user; generate an output based on a difference between the predicted physiological response and the user-estimated physiological response; and present the output to the user via a user interface.

In some aspects, the techniques described herein relate to a system, wherein the system comprises one or more image sensors configured to capture the one or more images during operation of the system by the user.

In some aspects, the techniques described herein relate to a system, wherein the system comprises an extended reality system.

In some aspects, the techniques described herein relate to a system, wherein the one or more consumables comprise at least one of one or more food items or one or more beverage items.

In some aspects, the techniques described herein relate to a system, wherein the physiological condition comprises blood glucose level.

In some aspects, the techniques described herein relate to a system, wherein the physiological condition comprises a metabolic state.

In some aspects, the techniques described herein relate to a system, wherein the physiological condition comprises one or more health metrics.

In some aspects, the techniques described herein relate to a system, wherein the user state information is determined based upon at least one of image data, sensor data, or user input.

In some aspects, the techniques described herein relate to a system, wherein the user state information indicates at least one of current or historic blood glucose level for the user.

In some aspects, the techniques described herein relate to a system, wherein the current or historic blood glucose level for the user is obtained via continuous glucose monitoring.

In some aspects, the techniques described herein relate to a system, wherein the user state information further indicates at least one of a ketone level, a lactate level, or an alcohol level for the user.

In some aspects, the techniques described herein relate to a system, wherein the user state information indicates one or more therapeutics administered to the user.

In some aspects, the techniques described herein relate to a system, wherein the user state information is determined based on image data that captures one or more physical components associated with the one or more therapeutics administered to the user.

In some aspects, the techniques described herein relate to a system, wherein the one or more therapeutics do not comprise insulin.

In some aspects, the techniques described herein relate to a system, wherein the one or more therapeutics are administered to the user within one or more threshold temporal proximities.

In some aspects, the techniques described herein relate to a system, wherein the user state information indicates at least one of a stress state or a disease state of the user.

In some aspects, the techniques described herein relate to a system, wherein the user state information indicates a blood alcohol level of the user.

In some aspects, the techniques described herein relate to a system, wherein the user state information indicates at least one of one or more financial preferences or one or more financial states of the user.

In some aspects, the techniques described herein relate to a system, wherein the user state information indicates one or more anticipated activities of the user.

In some aspects, the techniques described herein relate to a system, wherein the user state information indicates one or more user preferences associated with a lifestyle plan for the user.

In some aspects, the techniques described herein relate to a system, wherein the predicted physiological response comprises at least one of a predicted blood glucose level or a predicted blood glucose curve.

In some aspects, the techniques described herein relate to a system, wherein the predicted physiological response comprises a predicted metabolic state.

In some aspects, the techniques described herein relate to a system, wherein the predicted physiological response comprises a predicted effect on one or more health metrics.

In some aspects, the techniques described herein relate to a system, wherein determining the predicted physiological response further utilizes historical information associated with the user.

In some aspects, techniques described herein relate to a system, comprising: one or more processors; and one or more computer-readable recording media that store instructions that are executable by the one or more processors to configure the system to: access one or more images depicting one or more consumables, the consumption of which is able to influence a physiological condition of a user; access user state information associated with the user; determine a predicted physiological response to consumption of the one or more consumables by the user by using one or more models to process input based on at least the one or more images and the user state information; record one or more consumption choices of the user, the one or more consumption choices being associated with one or more timestamps; determine updated user state information, the updated user state information indicating one or more user states temporally subsequent to the one or more timestamps associated with the one or more consumption choices; and use the one or more consumption choices and the updated user state information to update the one or more models for determining predicted physiological responses to future consumption choices by the user.

In some aspects, the techniques described herein relate to a system, wherein the updated user state information indicates a measured physiological response of the user to the one or more consumption choices of the user.

In some aspects, the techniques described herein relate to a system, wherein updating the one or more models for determining predicted physiological responses to future consumption choices by the user further uses the predicted physiological response to consumption of the one or more consumables by the user.

In some aspects, the techniques described herein relate to a system, wherein the one or more consumption choices of the user are indicated by one or more additional images.

In some aspects, the techniques described herein relate to a system, wherein updating the one or more models for determining predicted physiological responses to future consumption choices by the user further uses the one or more additional images.

In some aspects, the techniques described herein relate to a system, wherein the one or more models comprise at least one of one or more machine learning models, one or more physiological models, one or more hybrid models, one or more control theory models, one or more agent-based models, one or more stochastic models, or one or more simulation models.

In some aspects, the techniques described herein relate to a system, wherein updating the one or more models for determining predicted physiological responses to future consumption choices by the user comprises: constructing training data using the one or more consumption choices and the updated user state information; and using the training data to train the one or more models.

In some aspects, the system described herein may comprise at least one input/output system and/or at least one communication system. In some aspects, the system described herein may comprise a mobile electronic device, a personal computing device, a mixed-reality head-mounted display, and/or an aerial vehicle.

### BRIEF DESCRIPTION OF THE DRAWINGS

To describe how the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered limiting in scope, embodiments will be described and explained with additional specificity and detail through the use of the accompanying drawings in which:

FIG. 1 illustrates example components of an example system that may include or be used to implement one or more disclosed embodiments.

FIGS. 2A, 2B, and 2C illustrate an example environment in which a user encounters consumables that may cause physiological responses and in which a system determines and/or processes predicted physiological responses.

FIG. 3 illustrates an example display of a user device depicting output generated based on the predicted physiological responses determined in association with the environment of FIGS. 2A, 2B, and 2C.

FIG. 4 illustrates the environment of FIGS. 2A, 2B, and 2C at a subsequent timepoint and illustrates the system determining and/or processing updated predicted physiological responses.

FIG. 5 illustrates the display of the user device depicting output generated based on the updated predicted physiological responses determined in association with the environment at the subsequent timepoint of FIG. 4.

FIGS. 6, 7, 8, and 9 illustrate example flow diagrams depicting acts associated with user physiological response prediction and management, in accordance with implementations of the present disclosure.

### DETAILED DESCRIPTION

Disclosed embodiments are generally directed to systems, methods, and apparatuses for facilitating physiological response prediction and/or management, such as by utilizing extended reality systems.

Individuals with diabetes face various challenges in maintaining stable blood sugar levels. At least some disclosed embodiments are directed toward automatically determining predicted physiological responses for users based on (i) an indication of a course of action and (ii) user state information. The indication of the course of action can be derived from numerous types of different sources and may take on various forms. For instance, an indication of a course of action may be an indication of one or more consumables (e.g., food or drink) that the individual may consume. The indication of the consumable(s) may be obtained based on image data (e.g., captured by one or more image sensors of a user device) or based on data received from a remote system (e.g., data pushed by a food provider system to a user system). Image data indicating the consumable(s) may include images captured of food or beverage items, images of labels or menus, and/or other types of images.

User state information may be based on sensor data, image data, user input, medical records, and/or other information. User state information may include, by way of example, blood glucose levels (current or historic, which may be obtained via continuous glucose monitoring (CGM)), other analyte levels (e.g., ketone, lactate or alcohol levels), therapeutics administered to the user (e.g., insulin and/or others), stress state, sleep adequacy, cardiovascular/other fitness metrics, disease state, blood alcohol level, anticipated activities, lifestyle preferences/plans, financial states/preferences, and/or others. User state information may comprise current states for users, previous or historical states/information for users, or a combination thereof.

The indication of the course of action and the user state information may be utilized as inputs to determine the predicted physiological response. The predicted physiological response may take on various forms, such as, by way of example, a predicted blood glucose level, a predicted blood glucose trend or curve (e.g., change in blood glucose level over time), a predicted metabolic state, a predicted effect on health metrics, and/or others. The predicted physiological response may be obtained by processing inputs using a statistical model or one or more machine learning or other type of artificial intelligence techniques (e.g., unsupervised learning, supervised learning, semi-supervised learning, reinformed learning, clustering, classification, regression, decision tree, neural networks, anomaly detection). An output may be generated and/or presented based on the predicted physiological response. The output may comprise, by way of example, a representation of the predicted physiological response, a predicted consequence of engaging in the course of action, a recommended action (e.g., an alternative course of action), comparative metrics (e.g., relative to an alternative predicted physiological response associated with an alternative course of action, including information related to past actions), and/or others. In some implementations, a system automatically reconfigures one or more devices (e.g., a therapeutic delivery device, such as an insulin pump) based on the predicted physiological response.

A predicted physiological response (and, consequently, output and/or device configurations) may be updated over time based on updated information related to a course of action and/or updated user state information. For example, updated image data capturing the state of consumption of a food or beverage item may be acquired and processed to update the predicted physiological response (e.g., via sensor data, user input). Similarly, as another example, updated user activity context (e.g., exercise) may be acquired (e.g., via sensor data, user input) to update the predicted physiological response (e.g., the predicted effect on blood glucose level from the initial course of action undertaken by the user).

Updated image data capturing the state of consumption of a food or beverage item may additionally or alternatively be used to automatically log consumption metrics (e.g., in a food log or meal log), which may help users manage compliance with lifestyle programs/goals/choices (e.g., diet programs, fitness programs, etc.).

In some implementations, a predicted physiological response may be utilized to provide users with insights and/or intuition related to the effects of various courses of action on user health outcomes and/or physiological responses. For instance, a system may output a prompt that enables a user to confirm a potential course of action (e.g., a user may provide input selecting a food or beverage product within their environment). The system may generate a predicted physiological response based on the potential course of action and may provide an output based on the predicted physiological response. In some instances, the system may provide output related to physiological responses automatically (e.g., based on a determined object of interest for a user, which may be based on user gaze location, focus duration, and/or other sensor-based information).

As another example, a system may prompt a user to provide a user-estimated physiological response to proceeding with a certain course of action. In response to receiving the user-estimated physiological response, the system may provide additional output based on a difference (if present) between the user-estimated physiological response and a system-estimated physiological response. Such activities may help users gain intuition for the effects that certain activities and/or lifestyle choices may have on their blood glucose level and/or other physiological states.

Although at least some examples herein are focused, in at least some respects, on implementations related to diabetes management, one will appreciate, in view of the present disclosure, that the principles discussed herein may be applied in other contexts and/or to other domains (e.g., alcohol consumption management, diet or fitness program compliance tracking, financial goal or lifestyle compliance tracking, and/or others).

### Example Systems and Components

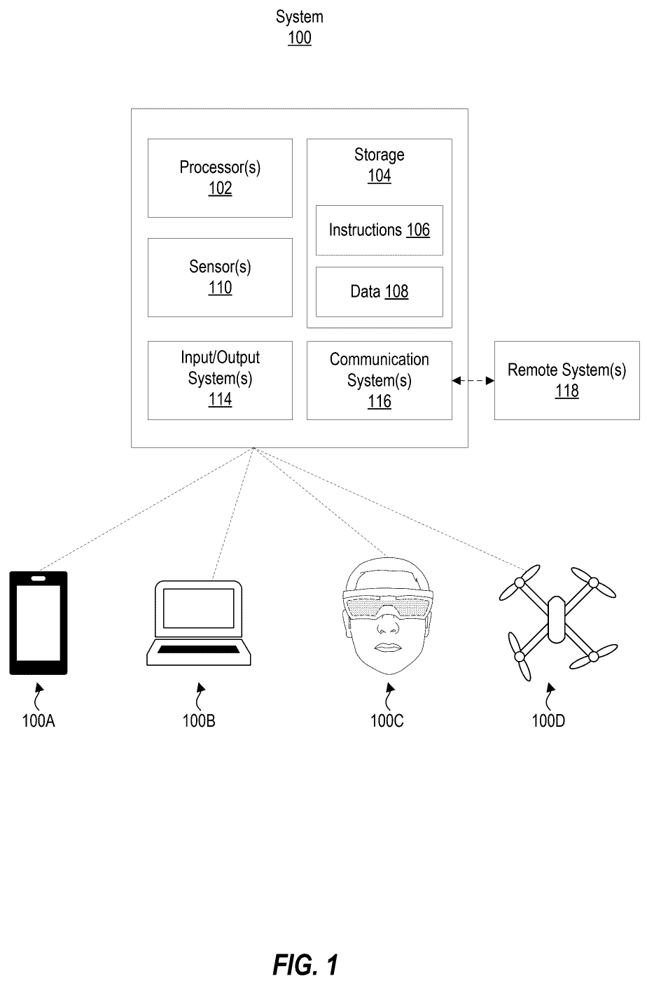

FIG. 1 illustrates various example components of a system 100 that may be used to implement one or more disclosed embodiments. For example, FIG. 1 illustrates that system 100 may include processor(s) 102, storage 104, sensor(s) 110, input/output system(s) 114 (I/O system(s) 114), and communication system(s) 116. Although FIG. 1 illustrates a system 100 as including particular components, one will appreciate, in view of the present disclosure, that system 100 may comprise any number of additional or alternative components.

The processor(s) 102 may comprise one or more sets of electronic circuitries that include any number of logic units, registers, and/or control units to facilitate the execution of computer-readable instructions (e.g., instructions that form a computer program). Such computer-readable instructions may be stored within storage 104. The storage 104 may comprise a computer-readable recording medium and may be volatile, non-volatile, or some combination thereof. Furthermore, storage 104 may comprise local storage, remote storage (e.g., accessible via communication system(s) 116 or otherwise), or some combination thereof. Additional details related to processors (e.g., processor(s) 102) and computer storage media (e.g., storage 104) will be provided hereinafter.

In some implementations, the processor(s) 102 may comprise or be configurable to execute any combination of software and/or hardware components that are operable to facilitate processing using machine learning models or other artificial intelligence-based structures/architectures. For example, processor(s) 102 may comprise and/or utilize hardware components or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

As will be described in more detail, the processor(s) 102 may be configured to execute instructions 106 stored within storage 104 to perform certain actions. The actions may rely at least in part on data 108 stored on storage 104 in a volatile or non-volatile manner.

In some instances, the actions may rely at least in part on communication system(s) 116 for receiving data from remote system(s) 118, which may include, for example, separate systems or computing devices, sensors, cloud resources, and/or others. The communications system(s) 116 may comprise any combination of software or hardware components that are operable to facilitate communication between on-system components/devices and/or with off-system components/devices. For example, the communications system(s) 116 may comprise ports, buses, or other physical connection apparatuses for communicating with other devices/components. Additionally, or alternatively, the communications system(s) 116 may comprise systems/components operable to communicate wirelessly with external systems and/or devices through any suitable communication channel(s), such as, by way of non-limiting example, Bluetooth, ultra-wideband, WLAN, infrared communication, and/or others.

FIG. 1 illustrates that system 100 may comprise or be in communication with sensor(s) 110. Sensor(s) 110 may comprise any device for capturing or measuring data representative of perceivable or detectable phenomena. By way of non-limiting example, the sensor(s) 110 may comprise one or more image sensors, microphones, thermometers, barometers, magnetometers, accelerometers, gyroscopes, inertial measurement units (IMUs), analyte sensors (e.g., CGM hardware, continuous lactate monitoring hardware, continuous ketone monitoring hardware, continuous alcohol monitoring hardware), force/stress sensors, electrode sensors, blood pressure monitors, continuous cortisol measurement hardware, and/or others. The sensor(s) 110 may comprise sensor systems/devices utilized to facilitate extended reality experiences and/or determine attributes/aspects of real-world objects (e.g., consumable characteristics), such as sensors for performing simultaneous localization and mapping (SLAM), depth sensors (e.g., active or passive stereo cameras, time-of-flight sensors, and/or others), gesture detection sensors, sound-based sensors, eye tracking sensors,

Furthermore, FIG. 1 illustrates that system 100 may comprise or be in communication with I/O system(s) 114. I/O system(s) 114 may include any type of input or output device such as, by way of non-limiting example, a touch screen, a mouse, a keyboard, a controller, and/or others, without limitation. For example, the I/O system(s) 114 may include a display system that may comprise any number of display panels, optics, laser scanning display assemblies, and/or other components.

FIG. 1 conceptually represents that the components of the system 100 may comprise or utilize various types of devices, such as mobile electronic device 100A (e.g., a smartphone), personal computing device 100B (e.g., a laptop), a mixed-reality head-mounted display 100C (HMD 100C), an aerial vehicle 100D (e.g., a drone), other devices (e.g., self-driving vehicles), combinations thereof, etc. System 100 may take on other forms in accordance with the present disclosure.

### Physiological Response Prediction and Management

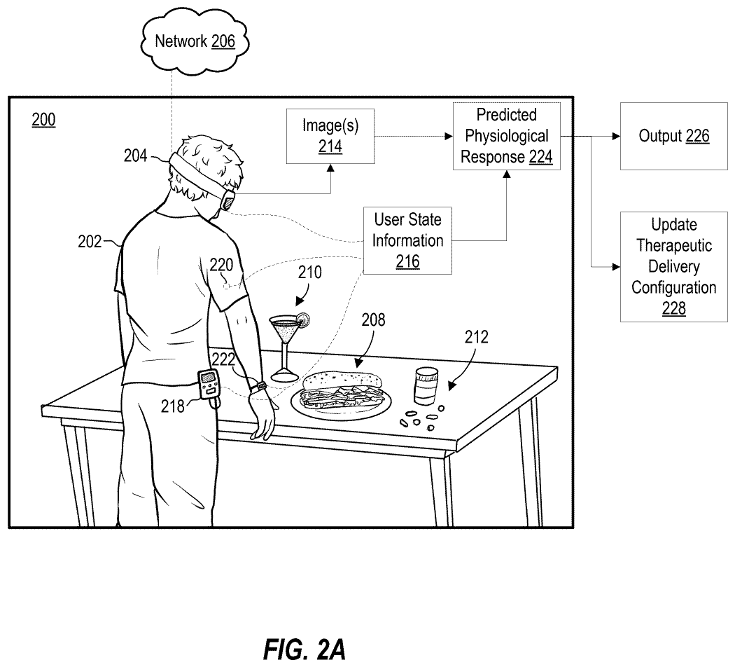

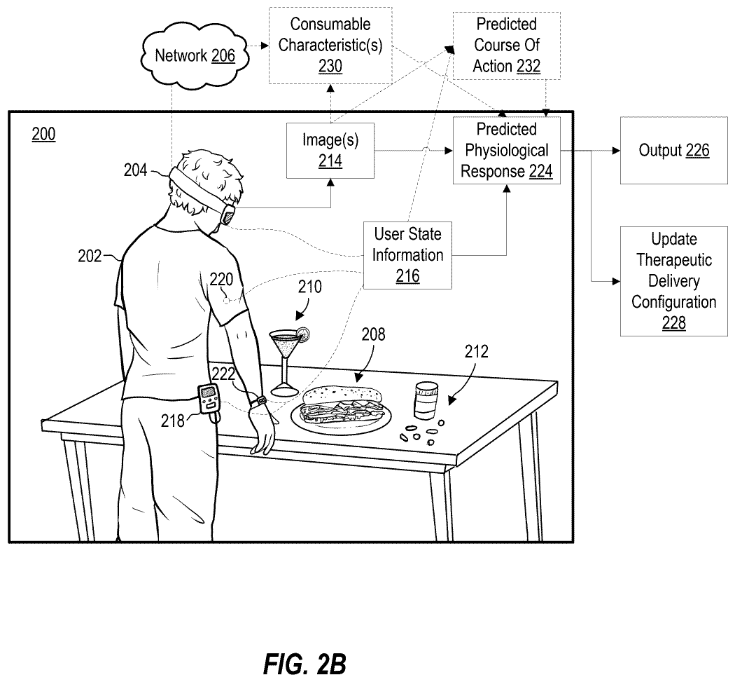

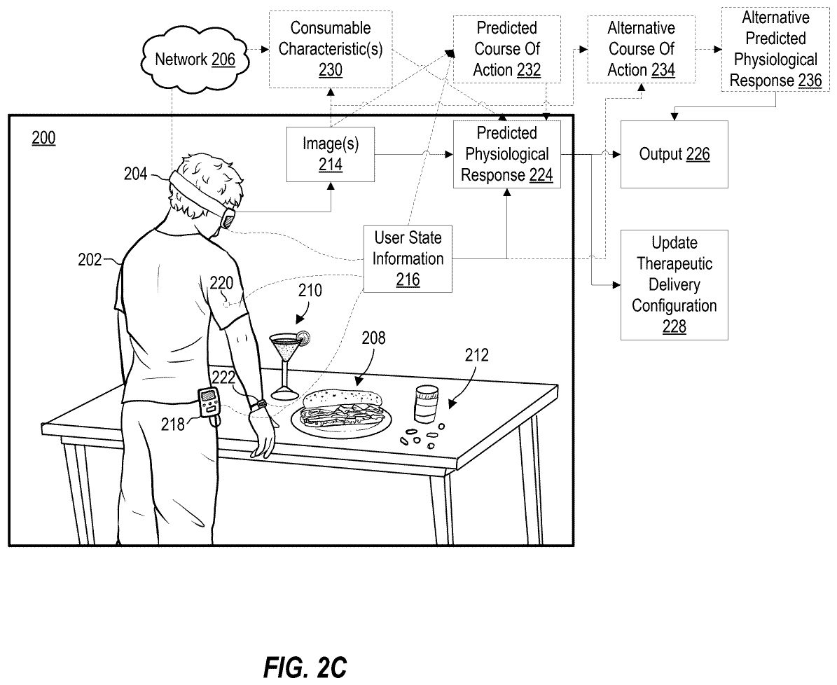

FIGS. 2A, 2B, and 2C illustrate an example environment 200 in which a user 202 encounters consumables that may influence a physiological condition of the user 202. The environment 200 includes a food item 208 (a sandwich), a beverage 210 (a cocktail), and a therapeutic 212 (medication in pill form). One will appreciate that the example combination of consumables shown in FIGS. 2A, 2B, and 2C is provided by way of illustrative example and for ease of description only, and that the consumption of certain beverages (e.g., alcoholic beverages) with certain therapeutics can be associated with significant risks. Such consumables may affect various types of physiological conditions, such as blood glucose level, metabolic states (e.g., postprandial state, ketogenic state, thermogenic state), and/or physiological conditions that may be represented by health metrics, such as body mass index (BMI), blood pressure, cholesterol level, resting heart rate, waist circumference or other measurements, fitness metrics (e.g., aerobic capacity, strength, flexibility, body composition), mental health, sleep quality, blood alcohol level (BAC), and/or others.

In the example of FIG. 2A, the user 202 has diabetes and thus faces challenges in maintaining stable blood glucose levels. Techniques will be described herein that may assist users in maintaining stable blood glucose levels. As noted hereinabove, although examples discussed herein focus, in at least some respects, on blood glucose level prediction and/or management, the principles discussed herein may be applied to other contexts (e.g., to facilitate prediction and/or managing of other types of physiological responses and/or conditions).

In the example of FIG. 2A, the user 202 operates a head-mounted display 204 (HMD 204), which corresponds to and includes one or more components of system 100 as discussed herein with reference to FIG. 1. For instance, the HMD 204 includes image sensors (e.g., sensor(s) 110) that enable the HMD 204 to capture images of objects in the environment 200 of the user 202 (e.g., while the user 202 operates the HMD 204 within the environment 200). The HMD 204 also comprises communication system(s) 116 that enable the HMD 204 to communicate with one or more other devices/systems via a network 206. The HMD 204 may comprise an extended reality HMD. As used herein, extended reality refers to augmented reality, virtual reality, mixed reality, and/or other immersive or semi-immersive modalities in which virtual content is presented to users. Although the example of FIG. 2A (and other Figures herein) focuses on utilizing an extended reality device (e.g., HMD 204) to perform various functions, other devices (e.g., a mobile electronic device, such as a smartphone, tablet, smartwatch, etc.) may be utilized in accordance with the principles described herein.

FIG. 2A conceptually depicts the HMD 204 capturing image(s) 214 of objects in the environment 200, in particular of the food item 208, the beverage 210, and the therapeutic 212. Although FIG. 2A depicts consumables in the form of actual, physical objects, consumables may be represented in other manners and may be captured by the image sensor(s) of the HMD 204. For instance, the HMD 204 may capture depictions of consumables (e.g., in printed materials such as menus or packaging, on displays, etc.), capture scannable elements associated with consumables (e.g., quick response (QR) codes, barcodes), capture text descriptions of consumables (e.g., in printed materials such as menus or packaging, on displays, etc.), and/or other information associated with consumables. The image(s) 214 (and/or information derived therefrom) may be utilized as an input to determine a predicted physiological response 224.

FIG. 2A also depicts user state information 216 associated with the user 202, which may also be utilized as an input to determine the predicted physiological response 224. The user state information 216 may comprise data related to the current or historic status of the user 202 in various aspects. The user state information 216 may be determined based upon image data, other types of sensor data, device data, user input, combinations thereof, and/or other types of data. By way of illustrative example, FIG. 2A illustrates an on-body sensor 220 (worn on the body of the user 202) that may be utilized to obtain current and/or historic blood glucose data for the user 202. Such blood glucose data may be represented in the user state information 216. Blood glucose data may be acquired via continuous glucose monitoring (CGM) or in other manners, such as via blood glucose measurements, user input at a device (manual input, voice input, gesture input, etc.), and/or other methods.

The on-body sensor 220 (or other sensor device) may acquire additional or alternative analyte measurements for the user such as ketone levels, lactate levels, alcohol levels, etc. (e.g., in a continuous manner). FIG. 2A depicts an additional on-body sensor device 222, which may comprise hardware for measuring various health metrics such as, by way of non-limiting example, heart rate, blood pressure, galvanic skin response (e.g., via electrodes), muscle tension (e.g., via electrodes or force/strain sensors), respiratory rate (e.g., via force/strain sensors), temperature, cortisol, activity states (e.g., via an accelerometer to determine exercise states, sleep states, etc.), and/or others.

Such analyte and/or other health metrics may be relevant in establishing a baseline or current state for the user 202, which may enable determination of the predicted physiological response 224 that may result from consuming the consumable(s) represented in the image(s) 214.

In some instances, the user state information 216 includes information related to a stress state or a disease state of the user 202. Such information may be acquired via sensor data (e.g., sensor data associated with muscle tension, blood pressure, heart rate, respiratory rate, temperature, and/or other metrics indicative of stress or disease state), record data (e.g., medical records/information associated with the user 202, which may be accessible via the network 206 or locally stored on the HMD 204), user input (e.g., provided by the user 202 to indicate stress level and/or diseases the user is experiencing), and/or other sources. In some instances, the sensor data, record data, or user input indicating stress state and/or disease state is obtained based on temporal relevance to the timepoint(s) associated with acquisition of the image(s) 214. For instance, upon acquisition of the image(s) 214, the HMD 204 may select sensor data indicating disease or stress state for the user 202 that are temporally proximate to the acquisition timepoint(s) of the image(s) 214, thereby allowing the system to identify stress or disease states for the user 202 that are likely to be present when the user 202 is likely to consume consumables represented in the image(s) 214.

Such disease or stress information may affect how consumption of the consumable(s) represented in the image(s) 214 affects the physiological response of the user 202. For example, liver or kidney disease, fevers, infections, inflammatory responses, or stress hormones may affect how a person with diabetes processes glucose. Such stress or disease states may thus be represented in the user state information 216 to enable the HMD 204 (or other system) to provide more robust predicted physiological responses 224.

Other bodily states or events may be relevant to the determination of a predicted physiological response 224, such as pregnancy, blood alcohol level, or therapeutics administered to or consumed by the user 202. Such bodily states or events may be acquired by a system (e.g., the HMD 204) and represented in the user state information 216 to facilitate determination of a predicted physiological response 224 (e.g., based on sensor data, user input, record data, etc.). For example, the HMD 204 may enable users to provide input indicating pregnancy status, blood alcohol level, or medication used. As another example, the system may capture image data (e.g., via the HMD 204) indicative of alcohol consumed by the user 202 (e.g., images capturing alcoholic beverages consumed by the user 202) or therapeutics administered to or taken by the user 202 (e.g., images capturing physical aspects/components of the therapeutics). For instance, the HMD 204 may capture imagery of the beverage 210 of FIG. 2A and identify it as an alcoholic beverage, and the HMD 204 may track consumption of the alcoholic beverage (e.g., estimated ounces consumed via sequentially captured imagery of the beverage) to indicate blood alcohol level for the user 202. As another example, the HMD 204 may be used to capture imagery of physical aspects of the therapeutics 212 (e.g., packaging, the pills themselves, labels, etc.) to classify the therapeutics, which may enable the system to determine the effect that the therapeutics 212 may have on the physiology of the user 202. For instance, based on image data, the therapeutics 212 may be determined to comprise corticosteroids, diuretics, or other medications that can affect blood glucose levels.

FIG. 2A also depicts a therapeutic delivery device 218 of the user 202, which is represented in FIG. 2A as an insulin pump. User state information 216 related to the administration of insulin (or other non-insulin therapeutics) may be obtained from operational data associated with the therapeutic delivery device 218.

Similar to the stress and/or disease state data, information related to other bodily states or events may be acquired for determination of a predicted physiological response 224 based on its temporal proximity to the expected consumption of consumables (e.g., which may be indicated by timestamps associated images of the consumables, or user input provided by the user 202 confirming consumption of or intent to consume the consumables). In some instances, the temporal proximity for selecting data to determine user state information 216 is depending on the type of data (e.g., blood alcohol may have a longer relevant temporal proximity than some therapeutics, and different therapeutics may have different durations of effect, which may enable different therapeutics to be associated with different temporal proximities based on therapeutic classification).

The user state information 216 may, in some implementations, capture information related to activity states of the user 202 that can affect physiological responses of the user. For instance, exercise, rest, or sleep activities can affect the blood glucose trend or curve (e.g., blood glucose levels of the user over time) of the user 202 and/or how quickly the user 202 processes glucose. In some instances, the user state information 216 may capture current or expected/anticipated user activity state or other user status (e.g., based on sensor data, calendar data, historical activity or location data for the user 202). Such information may be utilized to improve the accuracy of predicted physiological responses 224. For instance, user state information 216 may include location information associated with the user 202, and the location information may be utilized to determine available food options/alternatives (e.g., based on what restaurants/stores are nearby or available and nutrition information about the menu items) for recommendations or other output presented to the user 202.

In some instances, at least some of the user state information 216 is based on the particular consumable(s) represented in the image(s) 214. For instance, the user state information 216 may comprise historical information associated with previous physiological responses of the user 202 to previous instances of consuming consumables that are similar to those represented in the image(s) 214 (or other designation of consumables likely to be consumed or considered for consumption by the user 202). In some instances, user state information 216 is influenced by data stored in a reference database (e.g., a database storing physiological response information for groups of individuals).

FIG. 2A conceptually depicts the image(s) 214 and the user state information 216 being utilized as inputs (or utilized to generate inputs) to determine the predicted physiological response 224. The predicted physiological response 224 may be obtained by processing inputs derived from the image(s) 214 and the user state information 216 using a statistical or mathematical model or one or more machine learning or other type of artificial intelligence techniques (e.g., physiological/compartmental models such as minimal model, Hovorka model, or others; machine learning models such as random forest models, artificial neural networks, recurrent neural networks, long short-term memory (LSTM) networks, support vector machines, or others; hybrid models; control theory models such as proportional-integral-derivative control, model predictive control, or others; agent-based models, stochastic models, simulation models such as AIDA model, T1D simulator, or others; etc.). The parameters of such models or modules may be determined based on data that is specific to the user 202. In some implementations, the parameters of such models for determining the predicted physiological response 224 are trained and/or tuned based on actual measured physiological responses of the individual user 202 (e.g., after consumption or other activities). The determination of the predicted physiological response 224 (and/or the generation of the inputs for determining the predicted physiological response 224) may be performed at any suitable computing system, such as the HMD 204 or a cloud computing system that receives the inputs (and/or the image(s) 214 or the user state information 216) via the network 206.

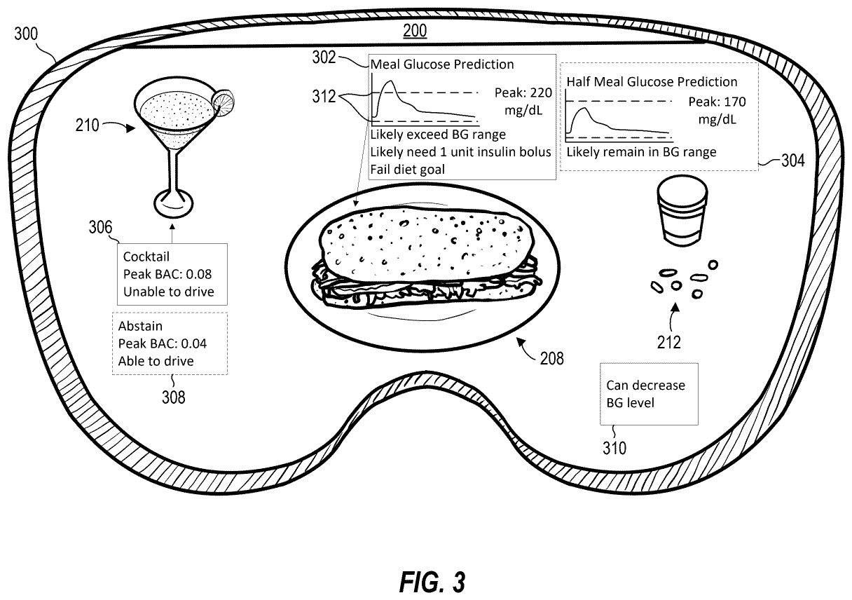

The predicted physiological response 224 may take on various forms, such as a predicted blood glucose level or blood glucose curve (e.g., how blood glucose levels may change over a period of time), a predicted metabolic state, a predicted effect on one or more health metrics, and/or others. FIG. 2A also conceptually depicts the predicted physiological response being used as a basis to generate an output 226 and/or to update a therapeutic delivery configuration 228. The output 226 may be presented on the HMD 204 and/or another user device, and/or may be recorded in a computer-readable recording medium (e.g., computer storage of the HMD 204 and/or another system accessible via the network 206). Additional details concerning the output 226 based on the predicted physiological response 224 will be provided hereinafter with reference to FIG. 3.

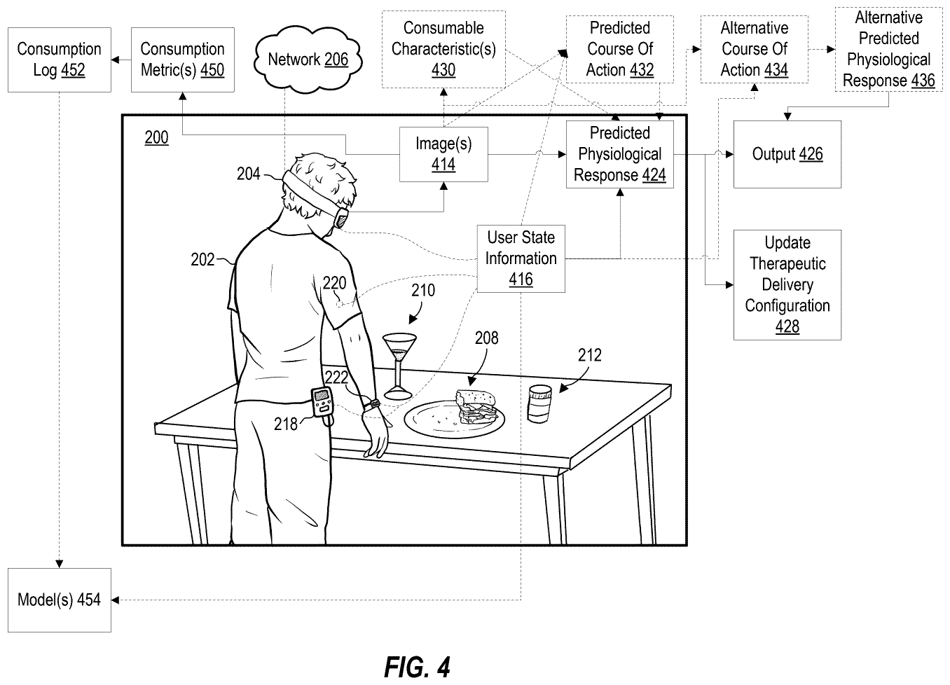

The act of updating a therapeutic delivery configuration 228 may involve, for instance, updating the therapeutic delivery configuration of the therapeutic delivery device 218 (e.g., the insulin pump). For instance, based on anticipated consumption by the user 202 and/or the predicted physiological response 224 associated therewith, the bolus or basal insulin configuration and/or the insulin delivery schedule of the insulin pump may be updated to compensate or account for the anticipated consumption by the user. Additionally, or alternatively, therapeutic delivery configurations may be updated based on confirmed consumption (e.g., confirmed by user input), as described hereinafter with reference to FIG. 4. Such functionality may help to prevent hyperglycemic or hypoglycemic events, and/or may improve time in range for the user 202.

In some instances, a system generates the predicted physiological response 224 by using input based on the image(s) 214 and/or the user state information 216 to generate intermediate classifications, predictions, and/or other labels, and by processing such intermediate classifications, predictions, and/or other labels to obtain the predicted physiological response 224. For instance, FIG. 2B conceptually depicts utilizing the image(s) 214 to obtain consumable characteristic(s) 230, which may comprise macronutrient profile, portion size, and/or other attributes of the consumable(s) represented in the image(s) 214. Consumable characteristic(s) 230 may be obtained by object segmentation/classification, optical character recognition (e.g., when the image(s) 214 depict a menu or written description of a food or beverage item), or other processing of the image(s) 214. FIG. 2B conceptually depicts the consumable characteristic(s) 230 used as an input to determine the predicted physiological response 224.

In some instances, consumable characteristic(s) 230 are obtained on the basis of information received from other devices (e.g., rather than image data captured by the HMD 204). For instance, FIG. 2B conceptually depicts the reception of an indication of one or more consumables over the network 206 to determine the consumable characteristic(s) 230 (represented by the dashed arrow from the network 206 to the consumable characteristic(s) 230 in FIG. 2B). In some implementations, the indication of the consumable(s) (and/or the characteristics thereof) is provided by an entity (e.g., a restaurant interface, a mobile application, an online ordering interface, a payment application) to enable determination of a predicted physiological response 224.

FIG. 2B also conceptually depicts determining a predicted course of action 232 as an intermediate representation for determining a predicted physiological response 224. For instance, input imagery of the food item 208 may cause generation of a predicted course of action 232 corresponding to consuming the food item 208. The predicted course of action 232 may additionally be based on user state information 216 (e.g., indicating historical behavior of the user 202 when confronted with similar consumption choices to those represented in the image(s) 214). The predicted course of action 232 may then be utilized as an input to determine the predicted physiological response 224.

In some implementations, the system refrains from generating intermediate classifications, predictions, and/or other labels when processing the input based on the image(s) 214 and/or the user state information 216 to determine the predicted physiological response 224.

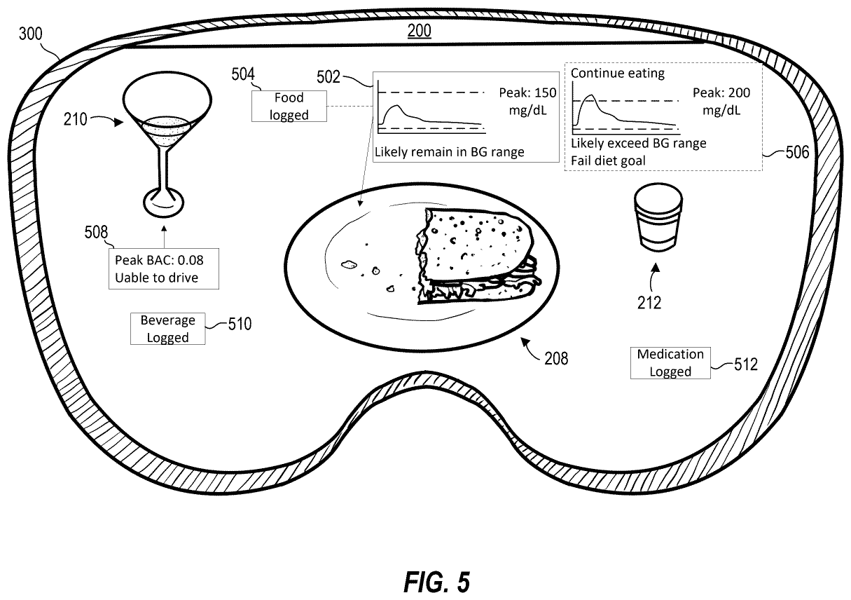

In some instances, as will be described in additional detail with reference to FIG. 3, the output 226 generated based on the predicted physiological response 224 is further based on an alternative predicted physiological response associated with an alternative course of action. For instance, FIG. 2C conceptually depicts the generation of an alternative course of action based on the image(s) 214 and/or the user state information 216. Continuing with the foregoing example, where the predicted course of action 232 corresponds to consumption of the food item 208, an alternative course of action 234 may comprise refraining from consuming the entire food item 208 (e.g., consuming half or another portion of the food item 208), modifying the food item 208 (e.g., refraining from adding a condiment to the food item, removing or substituting all or part of the food item such as the bun), consuming an alternative food item, taking compensatory action after consuming the food item (e.g., an exercise action, a therapeutic administration action) before or after consuming the food item, etc. The alternative course of action 234 may be selected based at least in part on the user state information 216, which may indicate lifestyle constraints and/or goals associated with the user 202. FIG. 2C furthermore depicts the alternative course of action 234 providing a basis for an alternative predicted physiological response 236, which may contribute to the output 226 (see FIGS. 3 and 5).

FIG. 3 illustrates an example display 300 of the HMD 204 from the perspective of the user 202. The display 300 depicts the environment 200 of the user and illustrates various example aspects of the output 226 generated based on the predicted physiological response 224 (and/or the alternative predicted physiological response 236). FIG. 3 depicts output elements 302, 304, 306, 308, and 310, which may comprise elements of the output 226 discussed above. The output elements 302, 304, 306, 308, and 310 of FIG. 3 are provided by way of illustrative example only. One will appreciate, in view of the present disclosure, that output 226 may take on any suitable form and/or may comprise additional or alternative components/features relative to those shown and described with reference to FIG. 3. Although FIG. 3 focuses on output 226 in the form of graphical representations, output 226 may be presented in additional or alternative formats, such as audio representations and/or haptic representations (e.g., utilizing speakers and/or haptic feedback devices). Furthermore, the display 300 may depict visual elements according to various modalities, such as by providing a direct view of one or more elements (e.g., where the display comprises a transparent material enabling users to view real-world objects, pursuant to an AR experience), providing virtual representations of one or more elements (e.g., where the display provides a pass-through representation of real-world objects, pursuant to a VR experience or an AR experience, or where the display provides virtual representations of output 226 pursuant to a VR experience or an AR experience), or a combination thereof.

FIG. 3 depicts the display 300 showing output element 302 in association with the food item 208. Output element 302 indicates various information based on the predicted physiological response 224 of the user 202 if the user proceeds with consuming the food item 208. In some instances, output element 302 is displayed in response to detection of the food item 208 in the image(s) 214 and/or in response to detection of one or more other conditions (e.g., determining that the food item 208 is an object of interest or intent to the user 202 based on gaze information, voice input, gesture input, or other factors).

In the example of FIG. 3, the output element 302 provides a representation of a predicted physiological response 224. For instance, output element 302 provides a predicted blood glucose curve and a peak blood glucose level (e.g., 220 mg/dl) that may result for the user 202 following consumption of the entire food item 208. Output element 302 of FIG. 3 also includes a representation of consequences that may result from proceeding with consuming the entire food item 208 (e.g., exceeding a user-specific blood glucose range, requiring a 1-unit insulin bolus to remain in range). Output element 302 may also provide range markers 312 (i.e., upper and lower dashed lines) indicating a desired glucose range for the user 202 to visually emphasize to users whether consumption behavior is likely to bring the user's glucose level outside of their desired glucose range. Providing a user with such information may assist the user 202 in making consumption choices that avoid adverse health consequences.

Output element 302 furthermore illustrates a consequence of consuming the food item 208 that relates to a lifestyle plan or goal of the user 202 (e.g., failing a diet goal). In some instances, the user state information 216 includes preferences of the user 202 associated with a lifestyle or other plan for the user, such as diet goals, exercise goals/regimens, or even financial preferences or states or goals. Based on the user state information 216, the output 226 may indicate how consumption (or selection for consumption, such as when selecting menu items) of one or more consumables may affect compliance of the user 202 with one or more goals, preferences, or other constraints.

Output element 304 includes a recommended action that the user 202 may choose to undertake as an alternative to proceeding with the predicted course of action associated with output element 302 (e.g., consuming the entire food item 208). The predicted course of action associated with output element 302 may correspond to the predicted course of action 232 discussed hereinabove with reference to FIG. 2B, and the recommended action associated with output element 304 may correspond to the alternative course of action 234 described hereinabove with reference to FIG. 2C. In the example of FIG. 3, the recommended action associated with output element 304 comprises consuming only half of the food item 208 (though other types of alternative courses of action are within the scope of the present disclosure, as described herein).

In the example of FIG. 3, the output element 304 provides a representation of an alternative predicted physiological response (236) associated with the recommended action of the output element 304 (e.g., consuming only half of the food item 208). Output element 304 provides a predicted alternative blood glucose curve and a peak blood glucose level (e.g., 170 mg/dl) that may result for the user 202 if the user 202 chooses to follow the recommended action. Output element 304 also provides a representation of consequences that may result from proceeding with the recommended action (e.g., remaining within blood glucose range). Providing a user with such comparative information associated with alternative courses of action may assist the user 202 in making consumption choices that avoid adverse health consequences.

Although output elements 302 and 304 focus, in at least some respects, on information associated with blood glucose levels, output 226 may comprise information related to other types of physiological responses, metabolic states, one or more health metrics, other analytes (e.g., ketone levels), and/or others. For instance, FIG. 3 also shows output element 306, which is displayed in association with the beverage 210 (e.g., an alcoholic beverage). Output element 306 indicates various information based on a predicted physiological response (224) of the user 202 if the user proceeds with consuming the beverage 210. In the example of FIG. 3, the output element 306 provides a representation of a predicted peak blood alcohol content of 0.08 that may result for the user 202 if the user proceeds with consuming the beverage 210.

Output element 306 also includes a representation of a consequence that may result from proceeding with consuming the beverage 210 (e.g., becoming legally unable to drive). In addition, more than one representation may be provided, for example when choosing different types of alcoholic beverage, in the context of both blood alcohol and glucose levels from the beverage's carbohydrate content. There may be information based on predicted levels of blood alcohol and glucose levels at different points in time since consuming the beverage, which may be separate blood alcohol and glucose level-based metrics or combined metrics such as ratio of peak blood alcohol content per grams of carbohydrate. Providing a user with such information may assist the user 202 in making consumption choices that avoid adverse consequences.

Output element 308 includes a recommended action that the user 202 may choose to undertake as an alternative to proceeding with the predicted course of action associated with output element 306 (e.g., consuming the beverage 210). In the example of FIG. 3, the recommended action associated with output element 308 includes abstaining from consuming the beverage 210. Output element 308 also provides a representation of an alternative predicted physiological response (236) associated with the recommended action of output element 308 (e.g., abstaining from consuming the beverage 210). The alternative predicted physiological response (236) of output element 308 includes a predicted peak blood alcohol content of 0.04. Output element 308 also provides a representation of consequences that may result from proceeding with the recommended action (e.g., remaining able to legally drive). Providing a user with such comparative information associated with alternative courses of action may assist the user 202 in making consumption choices that avoid adverse consequences.

FIG. 3 also demonstrates that output 226 may comprise other types of information and/or formats. For instance, FIG. 3 shows output element 310 in association with the therapeutics 212. Output element 310 indicates that consumption of the therapeutics 212 may cause a decrease in blood glucose levels. Output element 310 may be presented in response to detection of the therapeutics 212 in the image(s) 214 and/or in response to detection of one or more other conditions (e.g., determining that the therapeutics 212 are an object of interest or intent to the user 202 based on gaze information, voice input, gesture input, or other factors). Providing such information may provide opportunities for the user 202 to increase in understanding of the effects that lifestyle choices can have on blood glucose levels (or other physiological responses). Other types of output elements may be presented on an HMD 204 (or other device), such as educational content (e.g., related to the effects of consumption behavior, disease states, activity states, or other factors on physiological responses such as glucose patterns), activity-related content (e.g., guidance content for assisting users in administering therapeutics, exercising, or other activities), warnings (e.g., warning a user to not consume therapeutics 212 in conjunction with an alcoholic beverage 210 or when BAC is at certain levels, or warning a user to not consume an alcoholic beverage 210 while a therapeutic 212 is active, etc.), and/or other types of content. Such outputs may take on various forms as described herein, and/or others (e.g., in the form of a virtual avatar configured to present information, provide guidance, etc.).

In some instances, a system (e.g., the HMD 204) may prompt the user to provide input indicating a user-estimated physiological response associated with certain behavior (e.g., consuming consumables). In response to receiving input from a user indicating a user-estimated physiological response, the system may provide output based on a comparison or difference between the user-estimated physiological response and a system-predicted physiological response. Such functionality can additionally provide users with opportunities to increase in understanding of the effects that lifestyle choices can have on physiological responses.

FIG. 4 illustrates the environment 200 of FIGS. 2A, 2B, and 2C at one or more timepoints that are subsequent to the acquisition of image(s) 214 and/or the determination of the predicted physiological response 224. At the subsequent timepoint(s) shown in FIG. 4, the user has interacted with various consumables within the environment. In particular, FIG. 4 illustrates the food item 208 and the beverage as having been partially consumed by the user 202. FIG. 4 also shows the therapeutics 212 having been consumed by the user 202. In the example of FIG. 4, the HMD 204 captures subsequent image(s) 414 at the subsequent timepoint(s), such that the statuses of the consumables in the environment 200 after interaction by the user 202 are captured in the image(s) 414 (e.g., the portion(s) consumed thus far). Updated user state information 416 is also obtained, which may reflect changes to the state of the user 202 after at least partial consumption of the consumables within the environment 200. The updated image(s) 414 and user state information 416 may be used as a basis to determine an updated predicted physiological response 424 (intermediate determinations such as consumable characteristic(s) 430 and/or predicted course of action 432 may be utilized to determine the updated physiological response 424). The predicted physiological response 424 may be used to generate an updated output 426 and/or to further update a therapeutic delivery configuration 428 (e.g., for the therapeutic delivery device 218). The image(s) 414 and/or the user state information 416 may additionally be utilized to determine an alternative course of action 434 and/or an alternative predicted physiological response 436 associated therewith. The alternative predicted physiological response 436 may be represented in the updated output 426. The updated output 426 will be described in more detail with reference to FIG. 5.

FIG. 4 also conceptually depicts the image(s) 414 being used to determine consumption metric(s) 450, which may quantify the consumables that have been consumed by the user 202 (e.g., in terms of macronutrients/micronutrients consumed, vitamins/minerals consumed, number of calories, portion or weight of food/beverage, and/or other quantifications). The consumption metric(s) 450 may be recorded in a consumption log 452, which may track consumption of the user 202 over time (e.g., with accompanying timestamps) and may additionally or alternatively be used to update therapeutic delivery configurations of devices and/or to generate output for presentation to the user 202. For instance, the consumption log 452 may be regarded as an aspect of the user state information 416, such that updating the consumption log 452 with the consumption metric(s) 450 amounts to updating the user state information 416 with the consumption metric(s) 450. In some instances, consumption metric(s) 450 and/or additions to the consumption log 452 are confirmed by user input (e.g., by prompting the user 202 to confirm consumption).

In some implementations, the user state information 416, the consumption metric(s) 450, and/or the consumption log 452 are utilized to train, tune, or update one or more models 454 configured for determining the predicted physiological response 424 (e.g., as indicated in FIG. 4 by the dashed arrows extending from the consumption log 452 and the user state information 416 to the model(s) 454). By way of example, the consumption choices of the user 202 (e.g., consumption of the food item 208, the beverage 210, and/or the therapeutics 212, which may be represented in the consumption metric(s) 450, the consumption log 452, and/or the user state information 416) may be used in conjunction with blood glucose or other physiological response data of the user 202 following the consumption (e.g., represented in the user state information 416) as training data (or to construct training data). The training data may be used to train, tune, or updated the model(s) 454 (by training, tuning, or updating model parameters of the model(s) 454). The training data may be structured as a time-series with timestamps that includes event data (e.g., input variables) such as macronutrient intake, insulin dosage, physical activity, sleep, stress level, hormone/medical data, etc. (any of which may be represented in the user state information 416, consumption metric(s) 450, consumption log 452, etc.), paired with temporally subsequent glucose level or other physiological response data (e.g., output variables) to capture the effect of the event(s) on blood glucose or other physiological responses over time (e.g., time lags and/or lagged variables may be implemented to account for delayed responses). In some implementations, the image(s) 414 (and/or features, objects, or other information obtained therefrom or determined based thereupon) indicate the consumption choices of the user 202 and are used as part of the training data. The training data may be used in any suitable training method, such as supervised learning, backpropagation, parameter estimation, optimization, reinforcement learning, and/or others. As noted above, the model(s) 454 can take on various forms, such as physiological/compartmental models, machine learning models, hybrid models, control theory models, agent-based models, stochastic models, simulation models, and/or others.

FIG. 5 illustrates the display 300 of the user device (e.g., HMD 204) depicting various aspects of the updated output 426 generated based on the updated predicted physiological response 424 determined in association with the environment 200 at the subsequent timepoint(s) of FIG. 4. FIG. 5 illustrates output elements 502, 504, 506, 508, 510, and 512, which comprise elements of the updated output 426 discussed above. The output elements 502, 504, 506, 508, 510, and 512 of FIG. 5 are provided by way of illustrative example only. One will appreciate, in view of the present disclosure, that updated output 426 may take on any suitable form and/or may comprise additional or alternative components/features relative to those shown and described with reference to FIG. 5. Although FIG. 5 focuses on updated output 426 in the form of graphical representations, updated output 426 may be presented in additional or alternative formats, such as audio representations and/or haptic representations (e.g., utilizing speakers and/or haptic feedback devices).

FIG. 5 depicts the display 300 showing output element 502 in association with the food item 208, which has been partially consumed by the user 202. The partial consumption of the food item 208 by the user 202 may be reflected in the image(s) 414, the user state information 416 (e.g., in the consumption log 452), and/or may be confirmed by user input (e.g., FIG. 5 depicts output element 504, which confirms to the user 202 that partial consumption of the food item 208 has been logged within the consumption log 452). Output element 504 can additionally or alternatively indicate the amount/portion of the food item 208 consumed and/or an estimated peak blood glucose level (or other metric) based on the amount consumed.

In the example of FIG. 5, the output element 502 provides a representation of an updated predicted physiological response 424. For instance, output element 502 provides an updated predicted blood glucose curve and an updated predicted peak blood glucose level (e.g., 150 mg/dL) that may result from the user's consumption of the food item 208 up to the current timepoint (e.g., the portion(s) consumed). Output element 502 of FIG. 5 also includes a representation of consequences that may result from ceasing further consumption of the food item 208 (e.g., remaining within a user-specific blood glucose range). Providing a user with such information may assist the user 202 in making consumption choices that avoid adverse health consequences.

Output element 506 includes an alternative action that the user 202 may choose to undertake as an alternative to ceasing further consumption of the food item 208. In the example of FIG. 5, the output element 506 provides a representation of an alternative predicted physiological response (436) associated with the alternative action of the output element 506 (e.g., continuing to consume the rest of the food item 208). Output element 506 provides a predicted alternative blood glucose curve and a peak blood glucose level (e.g., 200 mg/dl) that may result for the user 202 if the user 202 chooses to follow the alternative action. Output element 506 also provides a representation of consequences that may result from proceeding with the alternative action (e.g., exceeding blood glucose range, failing a diet goal). Providing a user with such comparative information associated with alternative courses of action may assist the user 202 in making consumption choices that avoid adverse health consequences.

Although output elements 502 and 506 focus, in at least some respects, on information associated with blood glucose levels, the output 426 may comprise information related to other types of physiological responses, metabolic states, one or more health metrics, and/or others. For instance, FIG. 5 also shows output element 508, which is displayed in association with the beverage 210 (e.g., an alcoholic beverage). Output element 508 indicates various information based on an updated predicted physiological response (424) of the user 202 that may result from the user's partial consumption of the beverage 210. The partial consumption of the beverage 210 by the user 202 may be reflected in the image(s) 414, the user state information 416 (e.g., in the consumption log 452), and/or may be confirmed by user input (e.g., FIG. 5 depicts output element 508, which confirms to the user 202 that partial consumption of the beverage 210 has been logged within the consumption log 452). In the example of FIG. 5, the output element 508 provides a representation of the updated predicted peak blood alcohol content of 0.08 that will likely result for the user 202 in view of the user's consumption of the beverage 210. Output element 508 also includes a representation of a consequence that may result from the updated predicted physiological response (e.g., becoming legally unable to drive). Providing a user with such information may assist the user 202 in avoiding adverse occurrences.

FIG. 5 also demonstrates that updated output 426 may comprise other types of information and/or formats. For instance, FIG. 5 shows output element 512 in association with the therapeutics 212, which may confirm to the user that the user's consumption of the therapeutics 212 has been logged within the consumption log 452. Output element 512 can additionally or alternatively indicate the amount of therapeutics 212 consumed. Such output may assist users in avoiding over-consumption or under-consumption of medication (e.g., prevent missed doses or double doses) and/or may clearly indicate to users that medication consumption is captured by a system, which may increase user confidence that predictions/recommendations provided by the system are made with consideration toward consumed medication (or other consumables). FIG. 5 also shows output element 510 in association with the beverage 210, which may confirm to the user that the user's consumption of the beverage 210 has been logged within the consumption log 452. Output element 510 can additionally or alternatively indicate the amount/portion of beverage 210 that was consumed and/or an estimated peak BAC level based on the amount consumed.

Although display 300 of FIGS. 3 and 5 is described, in the examples above, as being presented on an HMD 204, a display 300 (or elements/components thereof, as described herein), may be presented on other types of devices or systems (e.g., systems 100, which may take on various forms as described hereinabove with reference to FIG. 1).

### Example Method(s)

The following discussion now refers to a number of methods and method acts that may be performed in accordance with the present disclosure. Although the method acts are discussed in a certain order and illustrated in a flow chart as occurring in a particular order, no particular ordering is required unless specifically stated, or required because an act is dependent on another act being completed prior to the act being performed. One will appreciate that certain embodiments of the present disclosure may omit one or more of the acts described herein.

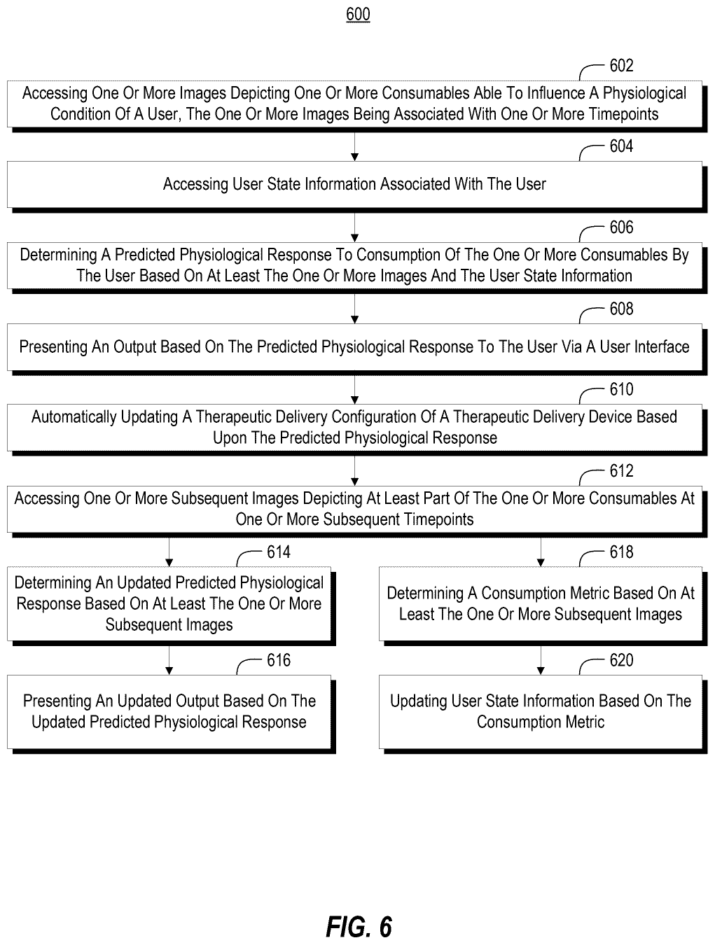

FIGS. 6, 7, 8, and 9 illustrate example flow diagrams 600, 700, 800, and 900, respectively, depicting acts associated with user physiological response prediction and management, in accordance with implementations of the present disclosure. In some instances, one or more acts of the illustrated flow diagrams may be performed utilizing one or more components of one or more systems as described herein (e.g., system 100, HMD 204, remote system(s) 118, combinations thereof, and/or others).

Act 602 of flow diagram 600 of FIG. 6 includes accessing one or more images depicting one or more consumables able to influence a physiological condition of a user, the one or more images being associated with one or more timepoints. In some instances, the one or more consumables comprise one or more food items or one or more beverage items. In some implementations, the physiological condition comprises blood glucose level. In some examples, the physiological condition comprises a metabolic state. In some instances, the physiological condition comprises one or more health metrics.

Act 604 of flow diagram 600 includes accessing user state information associated with the user. In some instances, the user state information is determined based upon image data, sensor data, or user input. In some implementations, the user state information indicates current or historic blood glucose level for the user. In some examples, the current or historic blood glucose level for the user is obtained via continuous glucose monitoring. In some instances, the user state information further indicates a ketone or lactate or alcohol level for the user. In some implementations, the user state information indicates one or more therapeutics administered to the user within one or more threshold temporal proximities to the one or more timepoints. In some examples, the user state information is determined based on image data that captures one or more physical components associated with the one or more therapeutics administered to the user. In some instances, the one or more therapeutics do not comprise insulin (e.g., analgesics, antipyretics, antihypertensives, anticoagulants, antidepressants, antipsychotics, antibiotics, antivirals, antifungals, antineoplastics, anti-inflammatory drugs, immunomodulators, antihistamines, bronchodilators, diuretics, antiemetics, anticonvulsants, antiretrovirals, hormone replacement drugs, antithyroid drugs, antianxiety drugs, cholesterol-lowering drugs, proton pump inhibitors, nonsteroidal anti-inflammatory drugs, muscle relaxants, beta-blockers, ace inhibitors, angiotensin ii receptor blockers, anticholinergics, etc.). In some implementations, the one or more threshold temporal proximities are determined based on one or more therapeutic classifications of the one or more therapeutics. In some examples, the user state information indicates a stress state or a disease state of the user. In some instances, the user state information indicates a blood alcohol level of the user. In some implementations, the user state information indicates one or more financial preferences or financial states of the user (e.g., budgeting/spending preferences, current or anticipated available financial resources, account balances, etc., which may affect consumption options recommended to a user). In some examples, the user state information indicates one or more anticipated activities of the user (e.g., based on sensor data, calendar data or scheduling data, historical activity or location data for a user, as described hereinabove with reference to FIG. 2). In some instances, the user state information indicates one or more user preferences associated with a lifestyle plan for the user (e.g., dietary goals/plans, exercise goals/plans, alcohol consumption goals/plans, as described hereinabove with reference to FIG. 2). The user state information may additionally or alternatively include activities of the user, such as exercise, rest, sleep, and/or others, which may be determined based on sensor data.

Act 606 of flow diagram 600 includes determining a predicted physiological response to consumption of the one or more consumables by the user based on at least the one or more images and the user state information. In some implementations, the predicted physiological response comprises a predicted blood glucose level or blood glucose curve. In some examples, the predicted physiological response comprises a predicted metabolic state. In some instances, the predicted physiological response comprises a predicted effect on one or more health metrics. In some implementations, determining the predicted physiological response further utilizes historical information associated with the user. In some examples, determining the predicted physiological response comprises determining one or more consumable characteristics associated with the one or more consumables and determining the predicted physiological response based on the one or more consumable characteristics. In some instances, determining the predicted physiological response comprises refraining from determining one or more consumable characteristics associated with the one or more consumables (e.g., by refraining from generating intermediate classifications, predictions, and/or other labels, such as when a system executes one or more artificial intelligence (AI) modules or machine learning (ML) models trained to infer physiological responses directly from image data and user state information without explicitly extracting or classifying consumption characteristics from the image data or the user state information).

Act 608 of flow diagram 600 includes presenting an output based on the predicted physiological response to the user via a user interface. In some implementations, the output comprises a graphical representation, an audio representation, or a haptic representation. In some examples, the output comprises a representation of the predicted physiological response. In some instances, the output comprises a predicted consequence of consuming the one or more consumables. In some implementations, the output comprises a recommended action. In some examples, the output is based on a comparison of the predicted physiological response and an alternative predicted physiological response associated with an alternative course of action.

Act 610 of flow diagram 600 includes automatically updating a therapeutic delivery configuration of a therapeutic delivery device based upon the predicted physiological response. In some instances, the therapeutic delivery device comprises an insulin pump operably connected to a system that performs one or more acts of flow diagram 600. In some implementations, the therapeutic delivery configuration comprises one or more of: bolus insulin configuration, basal insulin configuration, or insulin delivery schedule.

Act 612 of flow diagram 600 includes accessing one or more subsequent images depicting at least part of the one or more consumables at one or more subsequent timepoints. The one or more subsequent timepoints may be separated by any suitable temporal intervals (e.g., 1 minute, 5 minutes, 10 minutes, or any other temporal value less than, greater than, or between any of the foregoing values) and may reflect how users have interacted with various consumables in an environment, as described hereinabove with reference to FIG. 4. In some instances, user interaction with consumables may be reflected in subsequent images that capture containers, packaging, or other physical components associated with consumables.

Act 614 of flow diagram 600 includes determining an updated predicted physiological response based on at least the one or more subsequent images. In some examples, the updated predicted physiological response is further based on updated user state information associated with the user (e.g., user state information that reflects potential changes to the state of the user (see act 604) after at least partial consumption of one or more consumables, which may be based on sensor data, device data, or user input, as described hereinabove with reference to FIG. 4).

Act 616 of flow diagram 600 includes presenting an updated output based on the updated predicted physiological response (e.g., where the updated output depicts a representation of the updated predicted physiological response, an updated predicted blood glucose curve/trend/peak, updated consequences that may result from ceasing or continuing consumption, updated alternative actions that may be undertaken by a user, updated predicted alternative physiological responses, and/or others, as described hereinabove with reference to FIG. 5).

Act 618 of flow diagram 600 includes determining a consumption metric based on at least the one or more subsequent images. In some instances, act 618 may be performed concurrently or sequentially with acts 614 or 616.

Act 620 of flow diagram 600 includes updating user state information based on the consumption metric (e.g., by logging consumption of the consumable(s) within a consumption log, which may be utilized to influence future predictions/recommendations for a user and/or may be utilized to track compliance with lifestyle goals/plans and/or prescribed behavior for users, as described hereinabove with reference to FIG. 4). In some instances, act 620 may be performed concurrently or sequentially with acts 614 or 616.

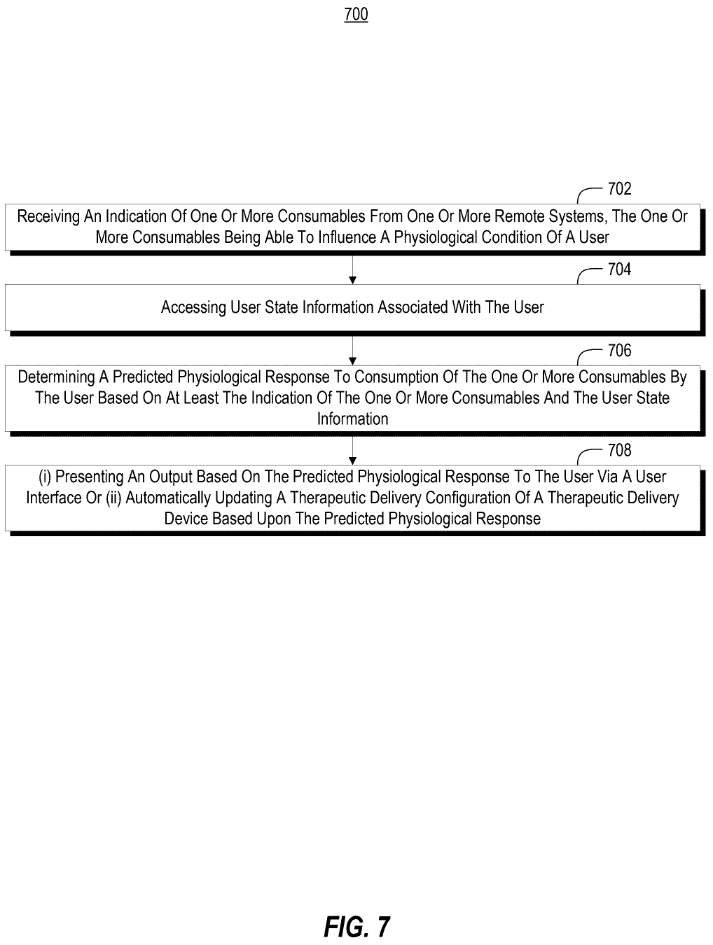

Turning to FIG. 7, act 702 of flow diagram 700 includes receiving an indication of one or more consumables from one or more remote systems, the one or more consumables being able to influence a physiological condition of a user. In some instances, the one or more consumables comprise one or more food items or one or more beverage items. In some implementations, the physiological condition comprises blood glucose level. In some examples, the physiological condition comprises a metabolic state. In some instances, the physiological condition comprises one or more health metrics.

Act 704 of flow diagram 700 includes accessing user state information associated with the user. In some implementations, the user state information is determined based upon image data, sensor data, or user input. In some examples, the user state information indicates current or historic blood glucose level for the user. In some instances, the current or historic blood glucose level for the user is obtained via continuous glucose monitoring. In some implementations, the user state information further indicates a ketone or lactate or alcohol level for the user. In some examples, the user state information indicates one or more therapeutics administered to the user. In some instances, the user state information is determined based on image data that captures one or more physical components associated with the one or more therapeutics administered to the user. In some implementations, the one or more therapeutics do not comprise insulin (e.g., analgesics, antipyretics, antihypertensives, anticoagulants, antidepressants, antipsychotics, antibiotics, antivirals, antifungals, antineoplastics, anti-inflammatory drugs, immunomodulators, antihistamines, bronchodilators, diuretics, antiemetics, anticonvulsants, antiretrovirals, hormone replacement drugs, antithyroid drugs, antianxiety drugs, cholesterol-lowering drugs, proton pump inhibitors, nonsteroidal anti-inflammatory drugs, muscle relaxants, beta-blockers, ace inhibitors, angiotensin ii receptor blockers, anticholinergics, etc.). In some examples, the one or more therapeutics are administered to the user within one or more threshold temporal proximities. In some instances, the user state information indicates a stress state or a disease state of the user. In some implementations, the user state information indicates a blood alcohol level of the user. In some examples, the user state information indicates one or more financial preferences or financial states of the user (e.g., budgeting/spending preferences, current or anticipated available financial resources, account balances, etc., which may affect consumption options recommended to a user). In some instances, the user state information indicates one or more anticipated activities of the user (e.g., anticipated exercise, rest, sleep, or other activity, based on sensor data, calendar data or scheduling data, historical activity or location data for a user, as described hereinabove with reference to FIG. 2). In some implementations, the user state information indicates one or more user preferences associated with a lifestyle plan for the user (e.g., dietary goals/plans, exercise goals/plans, alcohol consumption goals/plans, as described hereinabove with reference to FIG. 2). The user state information may additionally or alternatively include activities of the user, such as exercise, rest, sleep, and/or others, which may be determined based on sensor data.

Act 706 of flow diagram 700 includes determining a predicted physiological response to consumption of the one or more consumables by the user based on at least the indication of the one or more consumables and the user state information. In some examples, the predicted physiological response comprises a predicted blood glucose level or blood glucose curve. In some instances, the predicted physiological response comprises a predicted metabolic state. In some implementations, the predicted physiological response comprises a predicted effect on one or more health metrics. In some examples, determining the predicted physiological response further utilizes historical information associated with the user.

Act 708 of flow diagram 700 includes (i) presenting an output based on the predicted physiological response to the user via a user interface and (ii) automatically updating a therapeutic delivery configuration of a therapeutic delivery device based upon the predicted physiological response. In some instances, the output comprises a representation of the predicted physiological response. In some examples, the output comprises a predicted consequence of consuming the one or more consumables. In some implementations, the output comprises a recommended action. In some instances, the output is based on a comparison of the predicted physiological response and an alternative predicted physiological response associated with an alternative course of action. In some examples, the user interface comprises a display, a speaker, or a haptic feedback device. In some instances, the therapeutic delivery device comprises an insulin pump operably connected to a system that performs one or more acts of flow diagram 700. In some examples, the therapeutic delivery configuration comprises one or more of: bolus insulin configuration, basal insulin configuration, or insulin delivery schedule.

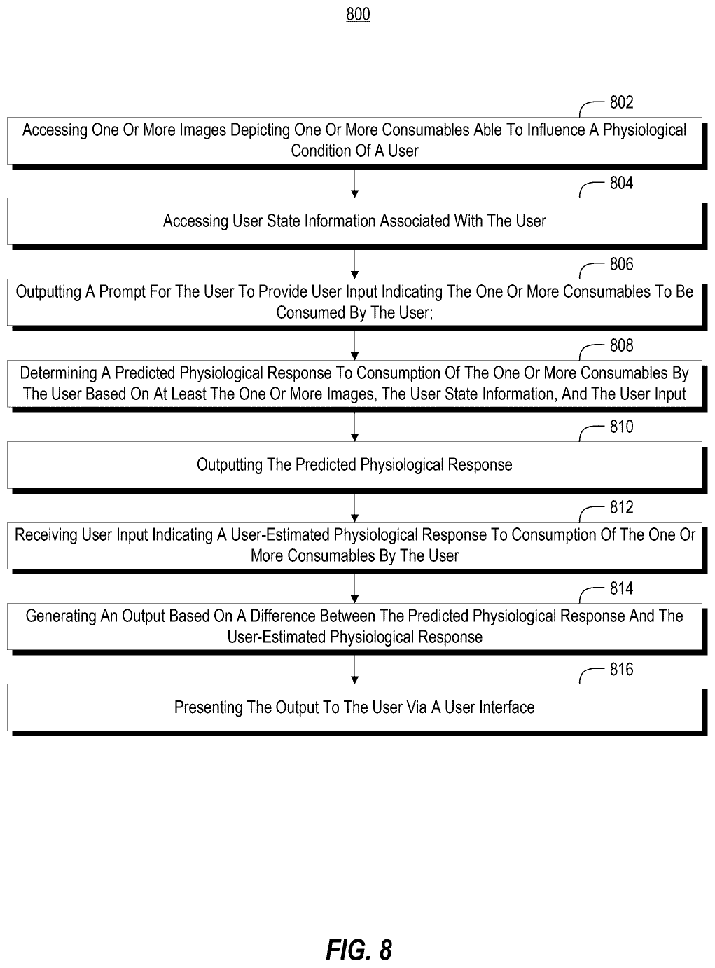

Turning to FIG. 8, act 802 of flow diagram 800 includes accessing one or more images depicting one or more consumables able to influence a physiological condition of a user. In some examples, the one or more consumables comprise one or more food items or one or more beverage items. In some implementations, the physiological condition comprises blood glucose level. In some instances, the physiological condition comprises a metabolic state. In some examples, the physiological condition comprises one or more health metrics.

Act 804 of flow diagram 800 includes accessing user state information associated with the user. In some implementations, the user state information is determined based upon image data, sensor data, or user input. In some instances, the user state information indicates current or historic blood glucose level for the user. In some examples, the current or historic blood glucose level for the user is obtained via continuous glucose monitoring. In some implementations, the user state information further indicates a ketone or lactate or alcohol level for the user. In some instances, the user state information indicates one or more therapeutics administered to the user. In some examples, the user state information is determined based on image data that captures one or more physical components associated with the one or more therapeutics administered to the user. In some implementations, the one or more therapeutics do not comprise insulin (e.g., analgesics, antipyretics, antihypertensives, anticoagulants, antidepressants, antipsychotics, antibiotics, antivirals, antifungals, antineoplastics, anti-inflammatory drugs, immunomodulators, antihistamines, bronchodilators, diuretics, antiemetics, anticonvulsants, antiretrovirals, hormone replacement drugs, antithyroid drugs, antianxiety drugs, cholesterol-lowering drugs, proton pump inhibitors, nonsteroidal anti-inflammatory drugs, muscle relaxants, beta-blockers, ace inhibitors, angiotensin ii receptor blockers, anticholinergics, etc.). In some instances, the one or more therapeutics are administered to the user within one or more threshold temporal proximities. In some examples, the user state information indicates a stress state or a disease state of the user. In some implementations, the user state information indicates a blood alcohol level of the user. In some instances, the user state information indicates one or more financial preferences or financial states of the user (e.g., budgeting/spending preferences, current or anticipated available financial resources, account balances, etc., which may affect consumption options recommended to a user). In some examples, the user state information indicates one or more anticipated activities of the user. In some implementations, the user state information indicates one or more user preferences associated with a lifestyle plan for the user. The user state information may additionally or alternatively include activities of the user, such as exercise, rest, sleep, and/or others, which may be determined based on sensor data.

Act 806 of flow diagram 800 includes outputting a prompt for the user to provide user input indicating the one or more consumables to be consumed by the user (e.g., where the user provides input indicating that the one or more consumables are an object of interest, consideration, or intent, as described hereinabove with reference to FIG. 3).

Act 808 of flow diagram 800 includes determining a predicted physiological response to consumption of the one or more consumables by the user based on at least the one or more images, the user state information, and the user input. In some instances, the predicted physiological response comprises a predicted blood glucose level or blood glucose curve. In some implementations, the predicted physiological response comprises a predicted metabolic state. In some examples, the predicted physiological response comprises a predicted effect on one or more health metrics. In some instances, determining the predicted physiological response further utilizes historical information associated with the user.

Act 810 of flow diagram 800 includes outputting the predicted physiological response (e.g., on a user interface, such as an extended reality display or other user device, in any suitable form such as a graphical representation, audio representation, haptic representation, as described hereinabove with reference to FIG. 3).

Act 812 of flow diagram 800 includes receiving user input indicating a user-estimated physiological response to consumption of the one or more consumables by the user (e.g., to provide users with a chance to test their understanding and/or intuition vis-à-vis the effect of consumption behavior on physiological outcomes, as described hereinabove with reference to FIG. 3).

Act 814 of flow diagram 800 includes generating an output based on a difference between the predicted physiological response and the user-estimated physiological response (e.g., to determine the magnitude of discrepancies between user-estimated physiological responses, which may indicate a level of user understanding of the relationship between consumption behavior and physiological responses, as described hereinabove with reference to FIG. 3).

Act 816 of flow diagram 800 includes presenting the output to the user via a user interface (e.g., to provide users with an opportunity to assess and/or fine-tune their understanding of the effects of their consumption behavior on physiological responses, as described hereinabove with reference to FIG. 3).

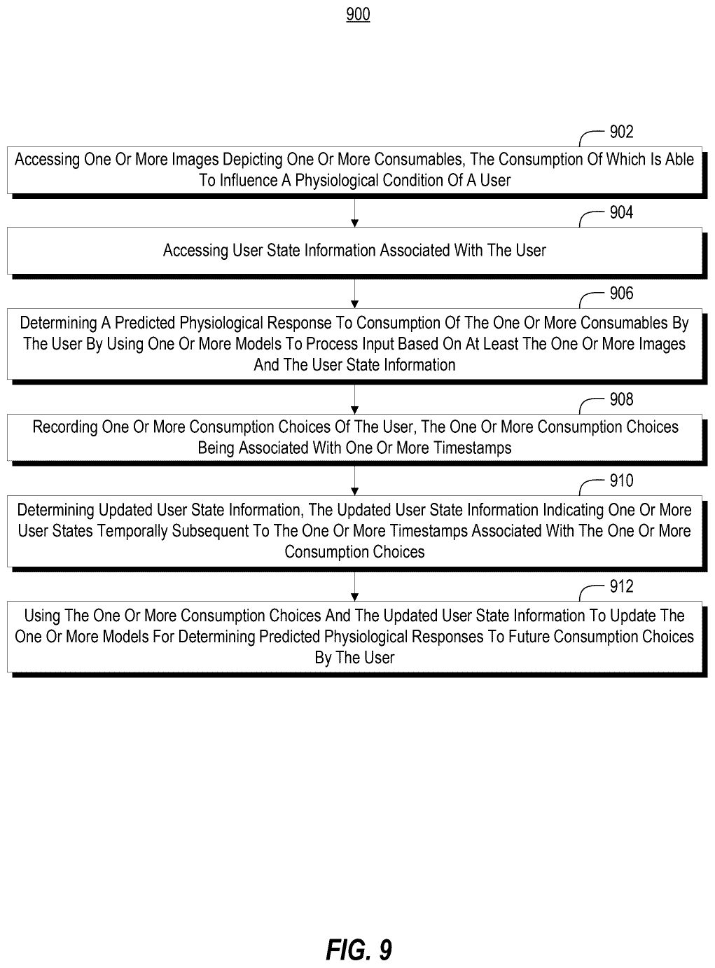

Act 902 of flow diagram 900 of FIG. 9 includes accessing one or more images depicting one or more consumables, the consumption of which is able to influence a physiological condition of a user.

Act 904 of flow diagram 900 includes accessing user state information associated with the user.

Act 906 of flow diagram 900 includes determining a predicted physiological response to consumption of the one or more consumables by the user by using one or more models to process input based on at least the one or more images and the user state information. In some instances, the one or more models comprise at least one of one or more machine learning models, one or more physiological models, one or more hybrid models, one or more control theory models, one or more agent-based models, one or more stochastic models, or one or more simulation models.

Act 908 of flow diagram 900 includes recording one or more consumption choices of the user, the one or more consumption choices being associated with one or more timestamps. In some implementations, the one or more consumption choices of the user are indicated by one or more additional images.

Act 910 of flow diagram 900 includes determining updated user state information, the updated user state information indicating one or more user states temporally subsequent to the one or more timestamps associated with the one or more consumption choices. In some examples, the updated user state information indicates a measured physiological response of the user to the one or more consumption choices of the user.

Act 912 of flow diagram 900 includes using the one or more consumption choices and the updated user state information to update the one or more models for determining predicted physiological responses to future consumption choices by the user. In some embodiments, updating the one or more models for determining predicted physiological responses to future consumption choices by the user further uses the predicted physiological response to consumption of the one or more consumables by the user. In some instances, updating the one or more models for determining predicted physiological responses to future consumption choices by the user further uses the one or more additional images. In some implementations, updating the one or more models for determining predicted physiological responses to future consumption choices by the user comprises: (i) constructing training data using the one or more consumption choices and the updated user state information; and (ii) using the training data to train the one or more models.

### Additional Details Related to the Disclosed Embodiments

Disclosed embodiments may comprise or utilize a special-purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “physical computer storage media” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links that can be used to carry program code in the form of computer-executable instructions or data structures, and which can be accessed by a general-purpose or special-purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special-purpose computer, or special-purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAS, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

One will also appreciate how any feature or operation disclosed herein may be combined with any one or combination of the other features and operations disclosed herein. Additionally, the content or feature in any one of the figures may be combined or used in connection with any content or feature used in any of the other figures. In this regard, the content disclosed in any one figure is not mutually exclusive and instead may be combinable with the content from any of the other figures.

As used herein, the term “about”, when used to modify a numerical value or range, refers to any value within 5%, 10%, 15%, 20%, or 25% of the numerical value modified by the term “about”.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

The invention is also defined in the following numbered clauses:

1. A system, comprising: one or more processors; and one or more computer-readable recording media that store instructions that are executable by the one or more processors to configure the system to: access one or more images depicting one or more consumables, the consumption of which is able to influence a physiological condition of a user; access user state information associated with the user; determine a predicted physiological response to consumption of the one or more consumables by the user based on at least the one or more images and the user state information; and present an output based on the predicted physiological response to the user via a user interface.

2. The system of clause 1, wherein the output comprises a representation of the predicted physiological response.

3. The system of clause 1 or 2, wherein the output comprises a predicted consequence of consuming the one or more consumables.

4. The system of clause 1, 2 or 3, wherein the output comprises a recommended action of the user.

5. The system of any preceding clause, wherein the output is based on a comparison of the predicted physiological response and an alternative predicted physiological response associated with an alternative course of action of the user.

6. The system of any preceding clause, wherein the system comprises one or more image sensors configured to capture the one or more images during operation of the system by the user.

7. The system of any preceding clause, wherein the system comprises an extended reality system.

8. The system of any preceding clause, wherein the one or more consumables comprise at least one of one or more food items or one or more beverage items.

9. The system of any preceding clause, wherein the physiological condition comprises blood glucose level.

10. The system of any preceding clause, wherein the physiological condition comprises a metabolic state.

11. The system of any preceding clause, wherein the physiological condition comprises one or more health metrics.

12. The system of any preceding clause, wherein the user state information is determined based upon at least one of image data, sensor data, and user input.

13. The system of any preceding clause, wherein the user state information indicates at least one of current and historic blood glucose level for the user.

14. The system of clause 13, wherein the current or historic blood glucose level for the user is obtained via continuous glucose monitoring.

15. The system of any of the preceding clauses, wherein the user state information further indicates at least one of a ketone level, a lactate level, or an alcohol level for the user.

16. The system of any preceding clause, wherein the one or more images are associated with one or more timepoints, and wherein the user state information indicates one or more therapeutics administered to the user within one or more threshold temporal proximities to the one or more timepoints.

17. The system of clause 16, wherein the user state information is determined based on image data that captures one or more physical components associated with the one or more therapeutics administered to the user.

18. The system of clause 16 or 17, wherein the one or more therapeutics do not comprise insulin.

19. The system of clause 16, 17 or 18, wherein the one or more threshold temporal proximities are determined based on one or more therapeutic classifications of the one or more therapeutics.

20. The system of any preceding clause, wherein the user state information indicates at least one of a stress state or a disease state of the user.

21. The system of any preceding clause, wherein the user state information indicates a blood alcohol level of the user.

22. The system of any preceding clause, wherein the user state information indicates one or more financial preferences or financial states of the user.

23. The system of any preceding clause, wherein the user state information indicates one or more anticipated activities of the user.

24. The system of any preceding clause, wherein the user state information indicates one or more user preferences associated with a lifestyle plan for the user.

25. The system of any preceding clause, wherein the predicted physiological response comprises at least one of a predicted blood glucose level or blood glucose curve.

26. The system of any preceding clause, wherein the predicted physiological response comprises a predicted metabolic state.

27. The system of any preceding clause, wherein the predicted physiological response comprises a predicted effect on one or more health metrics.

28. The system of any preceding clause, wherein the user interface comprises at least one of a display, a speaker, or a haptic feedback device of the system.

29. The system of any preceding clause, wherein the output comprises at least one of a graphical representation, an audio representation, or a haptic representation.

30. The system of any preceding clause, wherein the instructions are executable by the one or more processors to further configure the system to automatically update a therapeutic delivery configuration of a therapeutic delivery device based upon the predicted physiological response.

31. The system of clause 30, wherein the therapeutic delivery device comprises an insulin pump operably connected to the system.

32. The system of clause 30 or 31, wherein the therapeutic delivery configuration comprises one or more of: bolus insulin configuration, basal insulin configuration, or insulin delivery schedule.

33. The system of any preceding clause, wherein determining the predicted physiological response further utilizes historical information associated with the user.

34. The system of any preceding clause, wherein determining the predicted physiological response comprises determining one or more consumable characteristics associated with the one or more consumables and determining the predicted physiological response based on the one or more consumable characteristics.

35. The system of any of clauses 1 to 34, wherein the predicted physiological response is determined directly from the one or more images and the user state information without determining one or more consumable characteristics associated with the one or more consumables.

36. The system of any preceding clause, wherein the instructions are executable by the one or more processors to further configure the system to: access one or more subsequent images depicting at least part of the one or more consumables at one or more subsequent timepoints; determine an updated predicted physiological response based on at least the one or more subsequent images; and present an updated output based on the updated predicted physiological response.

37. The system of clause 12, wherein the updated predicted physiological response is further based on updated user state information associated with the user.

38. The system of any preceding clause, wherein the instructions are executable by the one or more processors to further configure the system to: access one or more subsequent images depicting at least part of the one or more consumables at one or more subsequent timepoints; determine a consumption metric based on at least the one or more subsequent images; and update user state information based on the consumption metric.

39. A system, comprising: one or more processors; and one or more computer-readable recording media that store instructions that are executable by the one or more processors to configure the system to: receive an indication of one or more consumables from one or more remote systems, consumption of the one or more consumables being able to influence a physiological condition of a user; access user state information associated with the user; determine a predicted physiological response to consumption of the one or more consumables by the user based on at least the indication of the one or more consumables and the user state information; and at least one of (i) present an output based on the predicted physiological response to the user via a user interface or (ii) automatically update a therapeutic delivery configuration of a therapeutic delivery device based upon the predicted physiological response.

40. The system of clause 39, wherein the output comprises a representation of the predicted physiological response.

41. The system of clause 39 or 40, wherein the output comprises a predicted consequence of consuming the one or more consumables.

42. The system of clause 39, 40 or 41, wherein the output comprises a recommended action of the user.

43. The system of any of clauses 39 to 42, wherein the output is based on a comparison of the predicted physiological response and an alternative predicted physiological response associated with an alternative course of action of the user.

44. The system of any of clauses 39 to 43, wherein the system comprises an extended reality system.

45. The system of any of clauses 39 to 44, wherein the one or more consumables comprise at least one of one or more food items or one or more beverage items.

46. The system of any of clauses 39 to 45, wherein the physiological condition comprises blood glucose level.

47. The system of any of clauses 39 to 46, wherein the physiological condition comprises a metabolic state.

48. The system of any of clauses 39 to 47, wherein the physiological condition comprises one or more health metrics.

49. The system of any of clauses 39 to 48, wherein the user state information is determined based upon at least one of image data, sensor data, and user input.

50. The system of any of clauses 39 to 49, wherein the user state information indicates at least one of current or historic blood glucose level for the user.

51. The system of clause 50, wherein the current or historic blood glucose level for the user is obtained via continuous glucose monitoring.

52. The system of any of clauses 39 to 51, wherein the user state information further indicates at least one of a ketone level, a lactate level or an alcohol level for the user.

53. The system of any of clauses 39 to 52, wherein the user state information indicates one or more therapeutics administered to the user.

54. The system of clause 53, wherein the user state information is determined based on image data that captures one or more physical components associated with the one or more therapeutics administered to the user.

55. The system of clause 53 or 54, wherein the one or more therapeutics do not comprise insulin.

56. The system of clause 53, 54 or 55, wherein the one or more therapeutics are administered to the user within one or more threshold temporal proximities.

57. The system of any of clauses 39 to 56, wherein the user state information indicates at least one of a stress state or a disease state of the user.

58. The system of any of clauses 39 to 57, wherein the user state information indicates a blood alcohol level of the user.

59. The system of any of clauses 39 to 58, wherein the user state information indicates at least one of one or more financial preferences or one or more financial states of the user.

60. The system of any of clauses 39 to 59, wherein the user state information indicates one or more anticipated activities of the user.

61. The system of any of clauses 39 to 60, wherein the user state information indicates one or more user preferences associated with a lifestyle plan for the user.

62. The system of any of clauses 39 to 61, wherein the predicted physiological response comprises at least one of a predicted blood glucose level or blood glucose curve.

63. The system of any of clauses 39 to 62, wherein the predicted physiological response comprises a predicted metabolic state.

64. The system of any of clauses 39 to 63, wherein the predicted physiological response comprises a predicted effect on one or more health metrics.

65. The system of any of clauses 39 to 64, wherein the user interface comprises at least one of a display, a speaker, or a haptic feedback device of the system.

66. The system of any of clauses 39 to 65, wherein the output comprises at least one of a graphical representation, an audio representation, or a haptic representation.

67. The system of any of clauses 39 to 66, wherein the therapeutic delivery device comprises an insulin pump operably connected to the system.

68. The system of clause 67, wherein the therapeutic delivery configuration comprises one or more of: bolus insulin configuration, basal insulin configuration, or insulin delivery schedule.

69. The system of any of clauses 39 to 68, wherein determining the predicted physiological response further utilizes historical information associated with the user.

70. A system, comprising: one or more processors; and one or more computer-readable recording media that store instructions that are executable by the one or more processors to configure the system to: access one or more images depicting one or more consumables the consumption of which is able to influence a physiological condition of a user; access user state information associated with the user; output a prompt for the user to provide user input indicating the one or more consumables to be consumed by the user; determine a predicted physiological response to consumption of the one or more consumables by the user based on at least the one or more images, the user state information, and the user input; and output the predicted physiological response.

71. The system of clause 70, wherein the instructions are executable by the one or more processors to further configure the system to: receive user input indicating a user-estimated physiological response to consumption of the one or more consumables by the user; generate an output based on a difference between the predicted physiological response and the user-estimated physiological response; and present the output to the user via a user interface.

72. The system of clause 70 or 71, wherein the system comprises one or more image sensors configured to capture the one or more images during operation of the system by the user.

73. The system of clause 70, 71 or 72, wherein the system comprises an extended reality system.

74. The system of any of clauses 70 to 73, wherein the one or more consumables comprise at least one of one or more food items or one or more beverage items.

75. The system of any of clauses 70 to 74, wherein the physiological condition comprises blood glucose level.

76. The system of any of clauses 70 to 75, wherein the physiological condition comprises a metabolic state.

77. The system of any of clauses 70 to 76, wherein the physiological condition comprises one or more health metrics.

78. The system of any of clauses 70 to 77, wherein the user state information is determined based upon at least one of image data, sensor data, or user input.

79. The system of any of clauses 70 to 78, wherein the user state information indicates at least one of current or historic blood glucose level for the user.

80. The system of clause 79, wherein the current or historic blood glucose level for the user is obtained via continuous glucose monitoring.

81. The system of any of clauses 70 to 80, wherein the user state information further indicates at least one of a ketone level, a lactate level, or alcohol level for the user.

82. The system of any of clauses 70 to 81, wherein the user state information indicates one or more therapeutics administered to the user.

83. The system of clause 82, wherein the user state information is determined based on image data that captures one or more physical components associated with the one or more therapeutics administered to the user.

84. The system of clause 82 or 83, wherein the one or more therapeutics do not comprise insulin.

85. The system of clause 82, 83 or 84, wherein the one or more therapeutics are administered to the user within one or more threshold temporal proximities.

86. The system of any of clauses 70 to 85, wherein the user state information indicates at least one of a stress state or a disease state of the user.

87. The system of any of clauses 70 to 86, wherein the user state information indicates a blood alcohol level of the user.

88. The system of any of clauses 70 to 87, wherein the user state information indicates at least one of one or more financial preferences or financial states of the user.

89. The system of any of clauses 70 to 88, wherein the user state information indicates one or more anticipated activities of the user.

90. The system of any of clauses 70 to 89, wherein the user state information indicates one or more user preferences associated with a lifestyle plan for the user.

91. The system of any of clauses 70 to 90, wherein the predicted physiological response comprises at least one of a predicted blood glucose level or a predicted blood glucose curve.

92. The system of any of clauses 70 to 91, wherein the predicted physiological response comprises a predicted metabolic state.

93. The system of any of clauses 70 to 92, wherein the predicted physiological response comprises a predicted effect on one or more health metrics.

94. The system of any of clauses 70 to 93, wherein determining the predicted physiological response further utilizes historical information associated with the user.

95. The system of any one of clauses 1, 39, or 70, wherein the predicted physiological response is determined via one or more machine learning models.

96. A system, comprising: one or more processors; and one or more computer-readable recording media that store instructions that are executable by the one or more processors to configure the system to: access one or more images depicting one or more consumables, the consumption of which is able to influence a physiological condition of a user; access user state information associated with the user; determine a predicted physiological response to consumption of the one or more consumables by the user by using one or more models to process input based on at least the one or more images and the user state information; record one or more consumption choices of the user, the one or more consumption choices being associated with one or more timestamps; determine updated user state information, the updated user state information indicating one or more user states temporally subsequent to the one or more timestamps associated with the one or more consumption choices; and use the one or more consumption choices and the updated user state information to update the one or more models for determining predicted physiological responses to future consumption choices by the user.

97. The system of clause 96, wherein the updated user state information indicates a measured physiological response of the user to the one or more consumption choices of the user.

98. The system of clause 96, or 97, wherein updating the one or more models for determining predicted physiological responses to future consumption choices by the user further uses the predicted physiological response to consumption of the one or more consumables by the user.

99. The system of any of clauses 96 to 98, wherein the one or more consumption choices of the user are indicated by one or more additional images.

100. The system of clause 96, wherein updating the one or more models for determining predicted physiological responses to future consumption choices by the user further uses the one or more additional images.

101. The system of any of clauses 96, to 100, wherein the one or more models comprise at least one of one or more machine learning models, one or more physiological models, one or more hybrid models, one or more control theory models, one or more agent-based models, one or more stochastic models, or one or more simulation models.

102. The system of any of clauses 96 to 101, wherein updating the one or more models for determining predicted physiological responses to future consumption choices by the user comprises: constructing training data using the one or more consumption choices and the updated user state information; and using the training data to train the one or more models.

103. The system of any one of clauses 1, 39, 70, or 96 further comprising at least one of one or more sensors, one or more input/output systems, or one or more communication systems.

We claim:

1. A system, comprising:
one or more processors; and
one or more computer-readable recording media that store instructions that are executable by the one or more processors to configure the system to:
access one or more images depicting one or more consumables, the consumption of which is able to influence a physiological condition of a user;
access user state information associated with the user;
determine a predicted physiological response to consumption of the one or more consumables by the user based on at least the one or more images and the user state information; and
present an output based on the predicted physiological response to the user via a user interface.

2. The system of claim 1, wherein the output is based on a comparison of the predicted physiological response and an alternative predicted physiological response associated with an alternative course of action of the user.

3. The system of claim 1, wherein the one or more images are associated with one or more timepoints, and wherein the user state information indicates one or more therapeutics administered to the user within one or more threshold temporal proximities to the one or more timepoints.

4. The system of claim 3, wherein the user state information is determined based on image data that captures one or more physical components associated with the one or more therapeutics administered to the user.

5. The system of claim 3, wherein the one or more therapeutics do not comprise insulin.

6. The system of claim 3, wherein the one or more threshold temporal proximities are determined based on one or more therapeutic classifications of the one or more therapeutics.

7. The system of claim 1, wherein the predicted physiological response comprises at least one of a predicted blood glucose level and blood glucose curve.

8. The system of claim 1, wherein the predicted physiological response comprises a predicted metabolic state.

9. The system of claim 1, wherein the instructions are executable by the one or more processors to further configure the system to automatically update a therapeutic delivery configuration of a therapeutic delivery device based upon the predicted physiological response.

10. The system of claim 9, wherein the therapeutic delivery device comprises an insulin pump operably connected to the system.

11. The system of claim 1, wherein the predicted physiological response is determined directly from the one or more images and user state information without determining one or more consumable characteristics associated with the one or more consumables.

12. The system of claim 1, wherein the instructions are executable by the one or more processors to further configure the system to:
access one or more subsequent images depicting at least part of the one or more consumables at one or more subsequent timepoints;
determine an updated predicted physiological response based on at least the one or more subsequent images; and
present an updated output based on the updated predicted physiological response.

13. The system of claim 12, wherein the updated predicted physiological response is further based on updated user state information associated with the user.

14. The system of claim 1, wherein the instructions are executable by the one or more processors to further configure the system to:
access one or more subsequent images depicting at least part of the one or more consumables at one or more subsequent timepoints;
determine a consumption metric based on at least the one or more subsequent images; and
update user state information based on the consumption metric.

15. A system, comprising:
one or more processors; and
one or more computer-readable recording media that store instructions that are executable by the one or more processors to configure the system to:
receive an indication of one or more consumables from one or more remote systems, consumption of the one or more consumables being able to influence a physiological condition of a user;
access user state information associated with the user;
determine a predicted physiological response to consumption of the one or more consumables by the user based on at least the indication of the one or more consumables and the user state information; and
at least one of (i) present an output based on the predicted physiological response to the user via a user interface, or (ii) automatically update a therapeutic delivery configuration of a therapeutic delivery device based upon the predicted physiological response.

16. The system of claim 15, wherein the output is based on a comparison of the predicted physiological response and an alternative predicted physiological response associated with an alternative course of action of the user.

17. The system of claim 15, wherein the user state information indicates one or more therapeutics administered to the user, and wherein the user state information is determined based on image data that captures one or more physical components associated with the one or more therapeutics administered to the user.

18. The system of claim 15, wherein the user state information indicates one or more therapeutics administered to the user, and wherein the one or more therapeutics are administered to the user within one or more threshold temporal proximities.

19. The system of claim 15, wherein the predicted physiological response comprises at least one of a predicted blood glucose level and blood glucose curve.

20. A system, comprising:
one or more processors; and
one or more computer-readable recording media that store instructions that are executable by the one or more processors to configure the system to:
access one or more images depicting one or more consumables consumption of which is able to influence a physiological condition of a user;
access user state information associated with the user;
output a prompt for the user to provide user input indicating the one or more consumables to be consumed by the user;
determine a predicted physiological response to consumption of the one or more consumables by the user based on at least the one or more images, the user state information, and the user input; and
output the predicted physiological response.
